(19) United States
(12) Patent Application Publication
(10) Pub. No.: US 2013/0165753 A1
(43) Pub. Date: Jun. 27, 2013

(54) IMAGE PROCESSING DEVICE, ENDOSCOPE APPARATUS, INFORMATION STORAGE DEVICE, AND IMAGE PROCESSING METHOD

(71) Applicant: OLYMPUS CORPORATION, Tokyo (JP)

(72) Inventor: Jumpei TAKAHASHI, Tokyo (JP)

(73) Assignee: OLYMPUS CORPORATION, Tokyo (JP)

(21) Appl. No.: 13/778,658

(22) Filed: Feb. 27, 2013

Related U.S. Application Data
(63) Continuation of application No. PCT/JP2011/068741, filed on Aug. 19, 2011.

(30) Foreign Application Priority Data
Sep. 9, 2010 (JP) 2010-201634

(51) Int. Cl. A61B 1/04; G06T 7/20

(52) U.S. Cl. 600/109; 382/128

(57) ABSTRACT

An image processing device processes an image acquired by an imaging section that enables magnifying observation. The image processing device includes a motion information acquisition section that acquires motion information that indicates a relative motion of the imaging section with respect to an object, an imaging magnification calculation section that calculates an imaging magnification of the imaging section, and an image extraction section that extracts an image within a specific area from a captured image acquired by the imaging section as an extracted image. The image extraction section sets the position of the specific area within the captured image based on the motion information, and sets the size of a margin area based on the imaging magnification and the motion information, the margin area being an area except the specific area in the captured image.

### CROSS REFERENCE TO RELATED APPLICATION

This application is a continuation of International Patent Application No. PCT/JP2011/068741, having an international filing date of Aug. 19, 2011 which designated the United States, the entirety of which is incorporated herein by reference. Japanese Patent Application No. 2010-201634 filed on Sep. 9, 2010 is also incorporated herein by reference in its entirety.

### BACKGROUND

The present invention relates to an image processing device, an endoscope apparatus, an information storage device, an image processing method, and the like.

An endoscope that can observe tissue at a magnification almost equal to that of a microscope (hereinafter referred to as “magnifying endoscope”) has been widely used for endoscopic diagnosis. The magnification of the magnifying endoscope is higher than that of a normal endoscope by a factor of several tens to several hundreds.

A pit pattern (microstructure) of the surface layer of the mucous membrane of tissue can be observed by utilizing the magnifying endoscope. The pit pattern of the surface layer of the mucous membrane of tissue differs between a lesion area and a normal area. Therefore, a lesion area and a normal area can be easily distinguished by utilizing the magnifying endoscope.

For example, JP-A-3-16470 discloses a method that implements shake canceling using a motion vector calculated from a plurality of images captured in time series.

### SUMMARY

According to one aspect of the invention, there is provided an image processing device that processes an image acquired by an imaging section that enables magnifying observation, the image processing device comprising:

a motion information acquisition section that acquires motion information that indicates a relative motion of the imaging section with respect to an object;

an imaging magnification calculation section that calculates an imaging magnification of the imaging section; and

an image extraction section that extracts an image within a specific area from a captured image acquired by the imaging section as an extracted image,

the image extraction section setting a position of the specific area within the captured image based on the motion information, and setting a size of a margin area based on the imaging magnification and the motion information, the margin area being an area except the specific area in the captured image.

According to another aspect of the invention, there is provided an endoscope apparatus comprising the above image processing device.

According to another aspect of the invention, there is provided a computer-readable storage device with an executable program stored thereon, wherein the program instructs a computer to perform steps of:

acquiring motion information that indicates a relative motion of an imaging section with respect to an object;

calculating an imaging magnification of the imaging section;

setting a position of a specific area within a captured image acquired by the imaging section based on the motion information, and setting a size of a margin area based on the imaging magnification and the motion information, the margin area being an area except the specific area in the captured image; and

extracting an image within the specific area from the captured image as an extracted image.

According to another aspect of the invention, there is provided an image processing method comprising:

acquiring motion information that indicates a relative motion of an imaging section with respect to an object;

calculating an imaging magnification of the imaging section;

extracting an image within a specific area from a captured image acquired by the imaging section as an extracted image; and

setting a position of the specific area within the captured image based on the motion information, and setting a size of a margin area based on the imaging magnification and the motion information, the margin area being an area except the specific area in the captured image.

### BRIEF DESCRIPTION OF THE DRAWINGS

FIG. 1 is a view illustrating a method according to one embodiment of the invention.

FIG. 2 is a view illustrating a method according to one embodiment of the invention.

FIG. 3 is a view illustrating a method according to one embodiment of the invention.

FIG. 4 is a view illustrating a method according to one embodiment of the invention.

FIG. 5 illustrates a configuration example of an endoscope apparatus.

FIG. 6 illustrates a configuration example of a color filter of a light source section.

FIG. 7 illustrates an example of the spectral transmittance characteristics of a color filter of a light source section.

FIG. 8 illustrates an example of the spectral sensitivity characteristics of an image sensor.

FIG. 9 illustrates a detailed configuration example of an image generation section.

FIG. 10 illustrates an example of an image acquired by an imaging section.

FIG. 11 illustrates an example of an image output from an image generation section.

FIG. 12 illustrates a detailed configuration example of an inter-channel motion vector detection section.

FIG. 13 illustrates a local area setting example during a block matching process.

FIG. 14 illustrates a detailed configuration example of an inter-frame motion vector detection section.

FIG. 15 is a view illustrating a magnification calculation method.

FIG. 16 illustrates a detailed configuration example of an image extraction section.

FIG. 17A is a view illustrating the size of a margin area, and FIG. 17B is a view illustrating the starting point coordinates of a margin area.

FIG. 18 is a view illustrating a clipping process.

FIG. 19 illustrates a second configuration example of an endoscope apparatus.

FIG. 20 illustrates an example of the spectral transmittance characteristics of a color filter of an image sensor.

FIG. 21 is a view illustrating a second magnification calculation method.

FIG. 22 illustrates a second detailed configuration example of an image extraction section.

FIG. 23 illustrates a third configuration example of an endoscope apparatus.

FIG. 24 illustrates a second detailed configuration example of an inter-frame motion vector detection section.

FIG. 25 illustrates a detailed configuration example of a magnification calculation section.

FIG. 26 is a view illustrating the definition of the moving direction of the end of an imaging section.

FIGS. 27A and 27B are views illustrating a third magnification calculation method.

FIG. 28 is a system configuration diagram illustrating the configuration of a computer system.

FIG. 29 is a block diagram illustrating the configuration of a main body included in a computer system.

FIG. 30 illustrates an example of a flowchart of a process according to one embodiment of the invention.

### DESCRIPTION OF EXEMPLARY EMBODIMENTS

According to one embodiment of the invention, there is provided an image processing device that processes an image acquired by an imaging section that enables magnifying observation, the image processing device comprising:

a motion information acquisition section that acquires motion information that indicates a relative motion of the imaging section with respect to an object;

an imaging magnification calculation section that calculates an imaging magnification of the imaging section; and

an image extraction section that extracts an image within a specific area from a captured image acquired by the imaging section as an extracted image,

the image extraction section setting a position of the specific area within the captured image based on the motion information, and setting a size of a margin area based on the imaging magnification and the motion information, the margin area being an area except the specific area in the captured image.

According to the image processing device, the relative motion information about the object and the imaging section is acquired, and the imaging magnification of the imaging section is calculated. The position of the specific area within the captured image is set based on the motion information, and the size of the margin area is set based on the imaging magnification. The extracted image is extracted from the captured image based on the position of the specific area and the size of the margin area. This makes it possible to implement shake canceling or the like corresponding to the imaging magnification.

Exemplary embodiments of the invention are described below. Note that the following exemplary embodiments do not in any way limit the scope of the invention laid out in the claims. Note also that all of the elements described in connection with the following exemplary embodiments should not necessarily be taken as essential elements of the invention.

### 1. Method

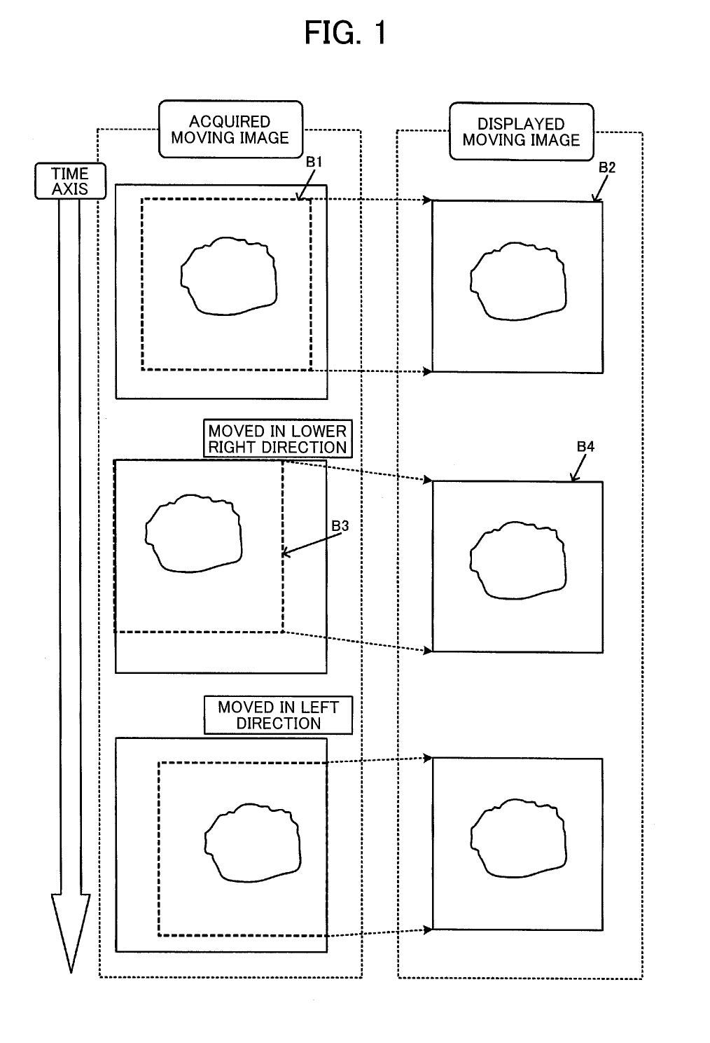

A shake canceling method is described below with reference to FIG. 1. As illustrated in FIG. 1, an area is extracted from a captured moving image (see B1), and an image of the extracted area is displayed (see B2). When the imaging section has moved in the lower right direction in the next frame (see B3), the image extraction target area is moved in the upper left direction, and an image of the same area as in the preceding frame is displayed (see B4). The relative shake between the imaging section and the object is canceled by thus moving the image extraction target area in the direction that cancels the shake.

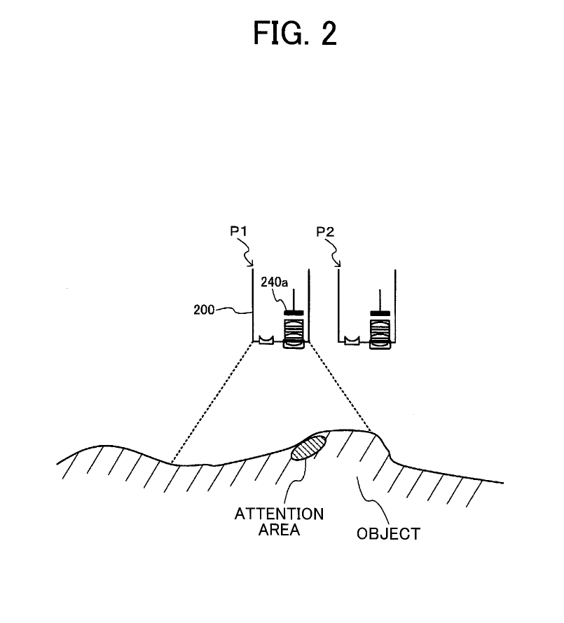

A problem that occurs when using the above shake canceling method is described below with reference to FIGS. 2 to 4. Note that the following description focuses on a G image for convenience of explanation.

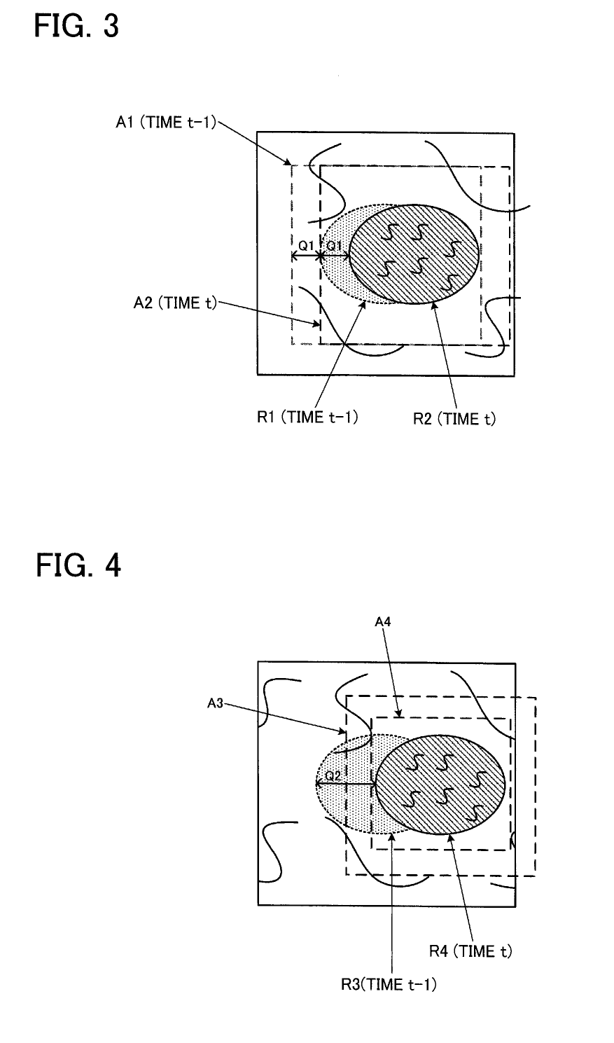

As illustrated in FIG. 2, the end of an imaging section 200 is situated at the position indicated by P1 at a time t−1. In this case, a G image that includes the attention area in the center area is acquired by an image sensor 240a (see R1 in FIG. 3). The image of the area indicated by A1 is used as the display image.

As illustrated in FIG. 2, the end of the imaging section 200 has moved to the position indicated by P2 at a time t. In this case, a G image that includes the attention area at a position shifted by Q1 is acquired by the image sensor 240a (see R2 in FIG. 3). The image of the area indicated by A2 that is shifted from the area indicated by A1 by the shift amount Q1 is used as the display image. The shift amount Q1 is detected by a known block matching process or the like using the G images acquired at the time t−1 and the time t.

The above process makes it possible to display a shake-canceled display image. However, the above method has a problem in that it is difficult to implement shake canceling when the shift amount Q1 is large. In FIG. 4, the attention area at the time t (see R4) is shifted to a large extent from the attention area at the time t−1 (see R3) by a shift amount Q2. In this case, part of the display image extraction target area is positioned outside the captured image (see A3). Since the right end of the area contains no image signal value, it is impossible to extract a display image.

Such a situation may occur during magnifying observation (diagnosis) using an endoscope, for example. Specifically, since the shift amount tends to increase in proportion to the magnification (imaging magnification) during magnifying observation, only a limited amount of shake can be canceled when applying the above shake canceling method directly to an endoscope apparatus.

In order to deal with the above problem, several embodiments of the invention employ a method that reduces the size of the area used as the display image corresponding to the magnification (see A4 in FIG. 4). This is equivalent to increasing the size of the area (margin area) that is not used as the display image corresponding to the magnification. Since the extraction target area is positioned within the captured image even if the shift amount is large by thus increasing the size of the margin area, it is possible to implement stable shake canceling even during magnifying observation.

### 2. Endoscope Apparatus

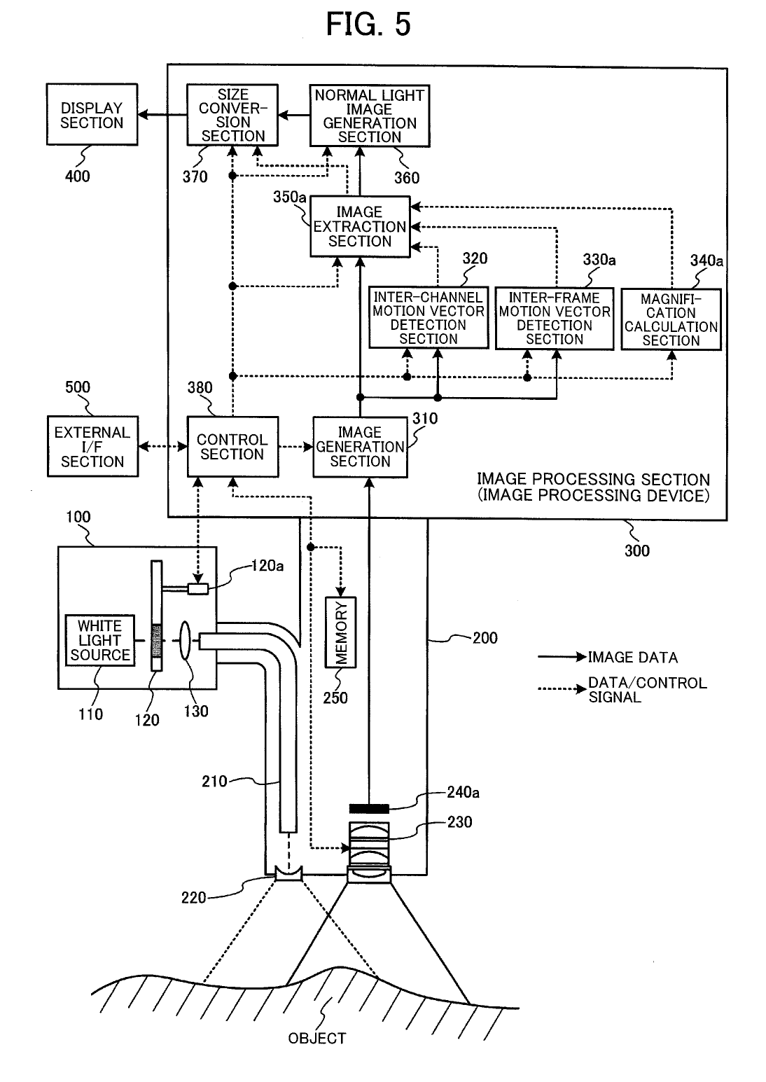

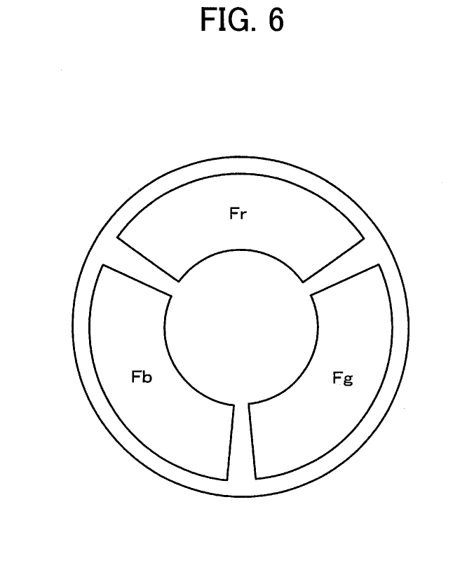

FIG. 5 illustrates a configuration example of an endoscope apparatus that changes the size of the margin area corresponding to the magnification. The endoscope apparatus (endoscope system) includes a light source section 100, an imaging section 200, an image processing section 300, a display section 400, and an external I/F section 500.

The light source section 100 includes a white light source 110 that emits white light, a rotary filter 120 that extracts light within a specific wavelength band from white light, a motor 120a that drives the rotary filter 120, and a lens 130 that focuses light extracted by the rotary filter 120 on a light guide fiber 210.

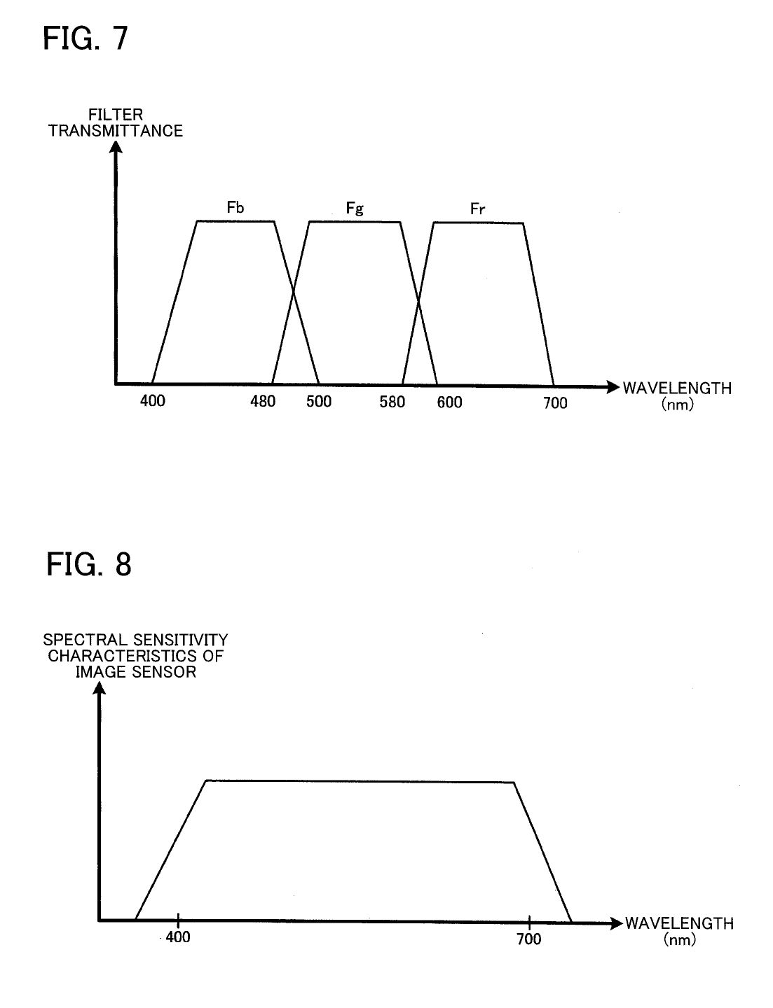

As illustrated in FIG. 6, the rotary filter 120 includes three color filters Fr, Fg, and Fb that differ in transmittance. As illustrated in FIG. 7, the color filter Fr allows light having a wavelength of 580 to 700 nm to pass through, the color filter Fg allows light having a wavelength of 480 to 600 nm to pass through, and the color filter Fb allows light having a wavelength of 400 to 500 nm to pass through, for example.

The motor 120a is bidirectionally connected to a control section 380. The rotary filter 120 is rotated by driving the motor 120a corresponding to a control signal output from the control section 380, so that the color filters Fg, Fr, and Fb are sequentially inserted into the optical path between the white light source 110 and the lens 130. The motor 120a outputs information about the color filter that is inserted into the optical path between the white light source 110 and the lens 130 to the control section 380. For example, the following identification information is used as the information about the color filter. The control section 380 outputs the identification information to an image generation section 310 (described later).

| Color filter inserted into optical path | Identification information |
|---|---|
| Fg | 1 |
| Fr | 2 |
| Fb | 3 |

The color filter is thus switched by rotating the rotary filter 120, and an image that corresponds to each color filter is captured by a monochrome image sensor 240a (described later). Specifically, an R image, a G image, and a B image are acquired in time series. The R image is acquired during a period in which the color filter Fr is inserted into the optical path, the G image is acquired during a period in which the color filter Fg is inserted into the optical path, and the B image is acquired during a period in which the color filter Fb is inserted into the optical path.

The imaging section 200 is farmed to be elongated and flexible (i.e., can be curved) so that the imaging section 200 can be inserted into a body cavity. The imaging section 200 is configured to be removable since a different imaging section 200 is used depending on the observation target part or the like. Note that the imaging section 200 is normally referred to as “scope” in the field of endoscopes. Therefore, the imaging section 200 is hereinafter appropriately referred to as “scope”.

The imaging section 200 includes the light guide fiber 210 that guides the light focused by the light source section 100, and an illumination lens 220 that diffuses the light that has been guided by the light guide fiber 210, and applies the diffused light to the object. The imaging section 200 also includes a condenser lens 230 that focuses reflected light from the object, and the image sensor 240a that detects the reflected light focused by the condenser lens 230. The image sensor 240a is a monochrome image sensor that has the spectral sensitivity characteristics illustrated in FIG. 8, for example.

The imaging section 200 further includes a memory 250. An identification number of each scope is stored in the memory 250. The memory 250 is connected to the control section 380. The control section 380 can identify the type of the connected scope by referring to the identification number stored in the memory 250.

The in-focus object plane position of the condenser lens 230 can be variably controlled. For example, the in-focus object plane position of the condenser lens 230 can be adjusted within the range of dmin to dmax (mm). For example, the user sets the in-focus object plane position d to an arbitrary value within the range of dmin to dmax (mm) via the external I/F section 500. The in-focus object plane position d set by the user via the external I/F section 500 is transmitted to the control section 380, and the control section 380 changes the in-focus object plane position of the condenser lens 230 by controlling the condenser lens 230 corresponding to the in-focus object plane position d set by the user. Note that the in-focus object plane position during normal (non-magnifying) observation is set to dn=dmax (mm).

The term “in-focus object plane position” used herein refers to the distance between the condenser lens 230 and the object when the object is in focus. The term “normal observation” used herein refers to observing the object in a state in which the in-focus object plane position is set to the maximum distance within the possible in-focus object plane position range, for example.

The in-focus object plane position control range differs depending on the connected scope. Since the control section 380 can identify the type of the connected scope by referring to the identification number of each scope stored in the memory 250, the control section 380 can acquire information about the in-focus object plane position control range dmin to dmax (mm) of the connected scope, and the in-focus object plane position dn during normal observation.

The control section 380 outputs the information about the in-focus object plane position to a magnification calculation section 340a (described later). The information output from the control section 380 includes information about the in-focus object plane position d set by the user, information about the in-focus object plane position dn during normal observation, and information about the minimum value dmin of the in-focus object plane position.

The image processing section 300 includes the image generation section 310, an inter-channel motion vector detection section 320, an inter-frame motion vector detection section 330a, the magnification calculation section 340a, an image extraction section 350a, a normal light image generation section 360, a size conversion section 370, and the control section 380. The control section 380 is connected to the image generation section 310, the inter-channel motion vector detection section 320, the inter-frame motion vector detection section 330a, the magnification calculation section 340a, the image extraction section 350a, the normal light image generation section 360, and the size conversion section 370, and controls the image generation section 310, the inter-channel motion vector detection section 320, the inter-frame motion vector detection section 330a, the magnification calculation section 340a, the image extraction section 350a, the normal light image generation section 360, and the size conversion section 370.

The image generation section 310 generates an RGB image from the R image, the G image, and the B image that have been acquired in time series by the image sensor 240a using a method described later. The inter-channel motion vector detection section 320 detects the motion vector between the RGB images generated by the image generation section 310. The inter-channel motion vector is the motion vector of the R image and the motion vector of the B image with respect to the G image.

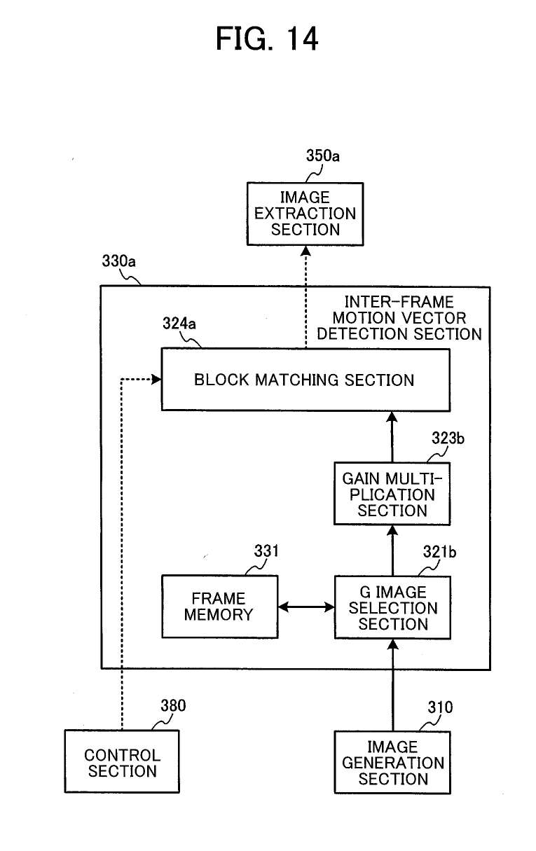

The inter-frame motion vector detection section 330a detects the inter-frame motion vector based on the RGB image in the preceding frame that is stored in a frame memory 331, and the RGB image output from the image generation section 310, as described later with reference to FIG. 14.

The magnification calculation section 340a calculates the magnification using the information about the in-focus object plane position output from the control section 380. Note that the magnification (imaging magnification) is the magnification of the object in the captured image, and is indicated by the relative ratio of the size of the imaging area on the object, for example. More specifically, when the magnification of an image obtained by capturing a reference imaging area is 1, the magnification of an image obtained by capturing an imaging area having a size half of that of the reference imaging area is 2.

The image extraction section 350a extracts an image from the RGB image output from the image generation section 310 based on the information output from the inter-channel motion vector detection section 320, the inter-frame motion vector detection section 330a, and the magnification calculation section 340a, and performs a shake canceling process. The image extraction section 350a outputs the extracted image as an R′G′B′ image. The image extraction section 350a outputs the ratio of the size of the RGB image to the size of the R′G′B′ image to the size conversion section 370. The details of the inter-channel motion vector detection section 320, the inter-frame motion vector detection section 330a, the magnification calculation section 340a, and the image extraction section 350a are described later.

The normal light image generation section 360 performs a white balance process, a color conversion process, a grayscale transformation process, and the like on the R′G′B′ image extracted by the image extraction section 350a to generate a normal light image.

The size conversion section 370 performs a size conversion process on the normal light image acquired by the normal light image generation section 360 so that the normal light image has the same size as that of the RGB image before extraction, and outputs the resulting image to the display section 400. More specifically, the size conversion section 370 performs a scaling process based on the ratio of the size of the RGB image to the size of the R′G′B′ image output from the image extraction section 350a. The scaling process may be implemented by a known bicubic interpolation process, for example.

### 3. Image Generation Section

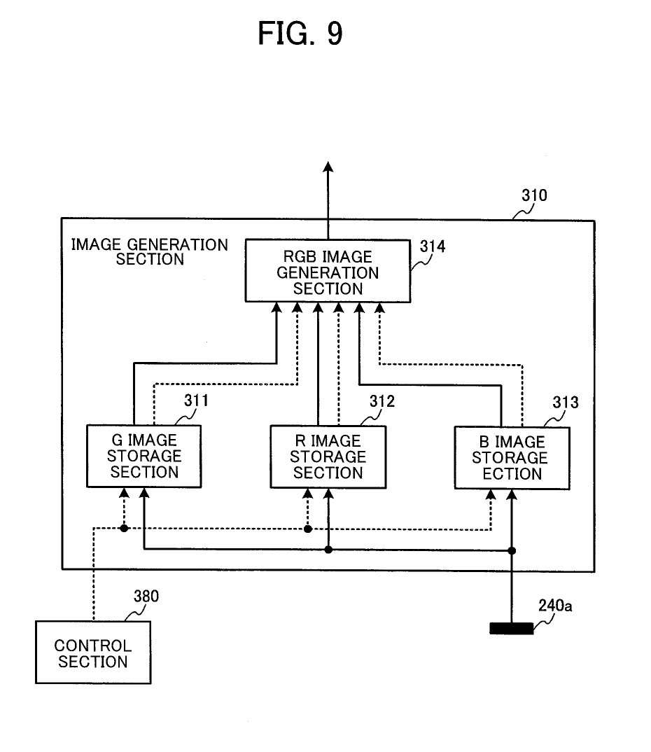

FIG. 9 illustrates a detailed configuration example of the image generation section 310. The image generation section 310 includes a G image storage section 311, an R image storage section 312, a B image storage section 313, and an RGB image generation section 314. The G image storage section 311, the R image storage section 312, and the B image storage section 313 are connected to the control section 380.

The G image storage section 311 refers to the identification information output from the control section 380, and determines a period in which the filter Fg is inserted into the optical path. More specifically, the G image storage section 311 determines that the filter Fg is inserted into the optical path when the identification information is “1”, and stores a signal output from the image sensor 240a as the G image during a period in which the filter Fg is inserted into the optical path.

The R image storage section 312 refers to the identification information output from the control section 380, and determines a period in which the filter Fr is inserted into the optical path. More specifically, the R image storage section 312 determines that the filter Fr is inserted into the optical path when the identification information is “2”, and stores a signal output from the image sensor 240a as the R image during a period in which the filter Fr is inserted into the optical path.

The B image storage section 313 refers to the identification information output from the control section 380, and determines a period in which the filter Fb is inserted into the optical path. More specifically, the B image storage section 313 determines that the filter Fb is inserted into the optical path when the identification information is “3”, and stores a signal output from the image sensor 240a as the B image during a period in which the filter Fb is inserted into the optical path. The G image storage section 311, the R image storage section 312, and the B image storage section 313 output a trigger signal to the RGB image generation section 314 after storing the image.

The RGB image generation section 314 reads the images stored in the G image storage section 311, the R image storage section 312, and the B image storage section 313 when the G image storage section 311, the R image storage section 312, or the B image storage section 313 has output the trigger signal, and generates the RGB image. The RGB image generation section 314 outputs the generated RGB image to the inter-channel motion vector detection section 320, the inter-frame motion vector detection section 330a, and the image extraction section 350a.

### 4. Inter-Channel Motion Vector Detection Section

The details of the inter-channel motion vector detection section 320 are described below. The inter-channel motion vector detection section 320 detects the motion vector of the B image and the motion vector of the R image with respect to the G image based on the RGB image output from the image generation section 310.

Specifically, since the R image, the G image, and the B image are acquired in time series, a color shift occurs in the RGB image acquired by the image generation section 310. Therefore, the motion vector of the B image and the motion vector of the R image with respect to the G image are detected by a block matching process. The color shift can be canceled by controlling the coordinates of each image extracted by the image extraction section 350a from the RGB image corresponding to the detected motion vector.

An R image captured by an endoscope does not contain sufficient structural information (e.g., blood vessel). Therefore, it is difficult to detect the motion vector of the R image using the block matching process. In one embodiment of the invention, the motion vector of the B image is detected using the block matching process, and the motion vector of the R image is estimated from the motion vector of the B image, as described later.

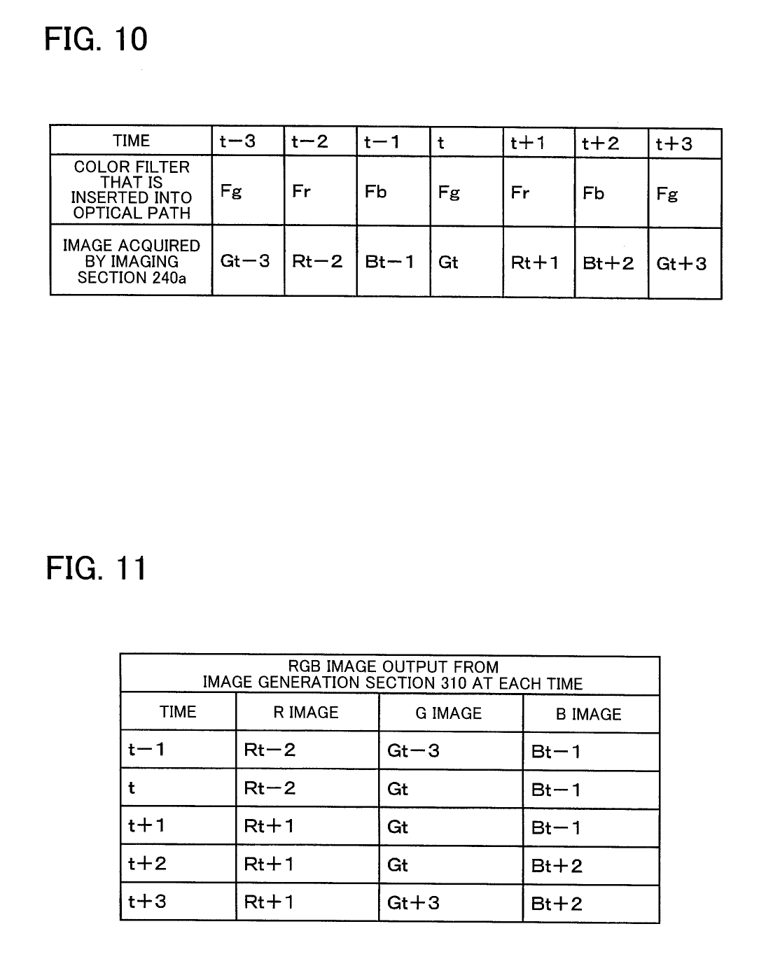

The inter-channel motion vector detection section 320 is described in detail below with reference to FIGS. 10 to 13. As illustrated in FIG. 10, the imaging section 200 acquires an image at times t−3 to t+3. In this case, an R image, a G image, and a B image illustrated in FIG. 11 are output as the RGB image that is output from the image generation section 310 at each time.

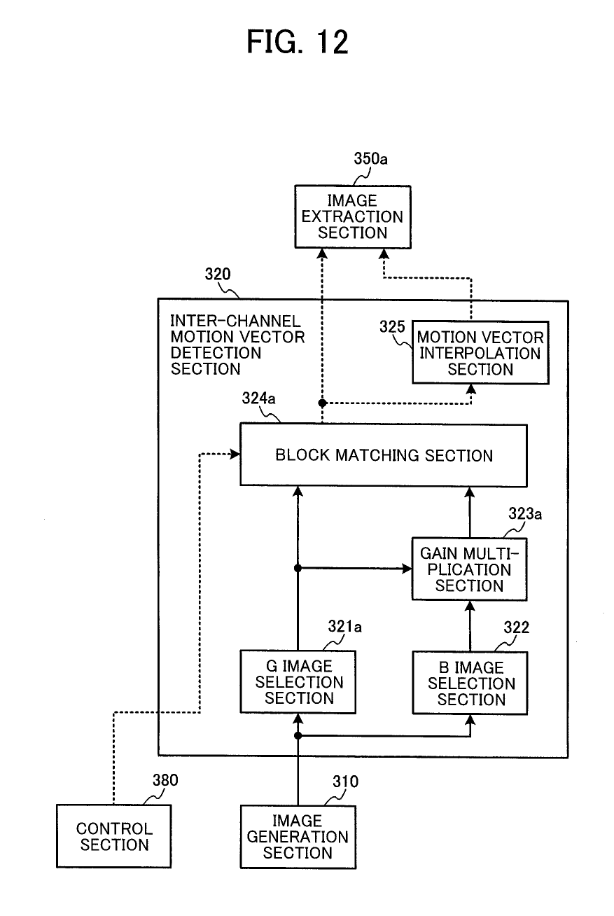

FIG. 12 illustrates a detailed configuration example of the inter-channel motion vector detection section 320. The inter-channel motion vector detection section 320 includes a G image selection section 321a, a B image selection section 322, a gain multiplication section 323a, a block matching section 324a, and a motion vector interpolation section 325. The block matching section 324a is connected to the control section 380.

The G image selection section 321a selects the G image from the RGB image output from the image generation section 310, and outputs the G image to the gain multiplication section 323a and the block matching section 324a. The B image selection section 322 selects the B image from the RGB image output from the image generation section 310, and outputs the B image to the gain multiplication section 323a.

The gain multiplication section 323a multiplies each pixel of the B image by a gain so that the average signal value of the B image is equal to the average signal value of the G image. The gain multiplication section 323a outputs the B image that has been multiplied by the gain to the block matching section 324a. More specifically, the gain multiplication section 323a calculates the gain “gain” using the following expression (1). Note that G_ave indicates the average signal value of the entire G image, and B_ave indicates the average signal value of the entire B image.

gain=G_ave/B_ave (1)

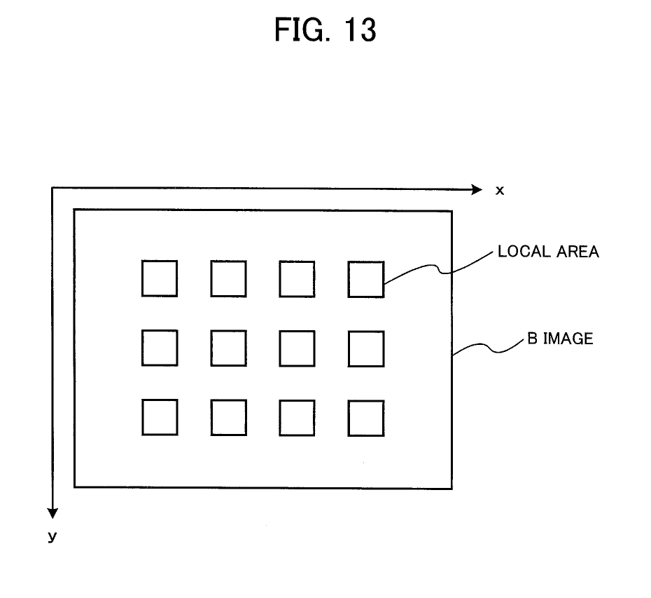

The block matching section 324a sets a plurality of local areas to the B image output from the gain multiplication section 323a. FIG. 13 illustrates a local area setting example. In one embodiment of the invention, the xy coordinate system illustrated in FIG. 13 is used as the coordinate system of the image. The xy coordinates are two-axis orthogonal coordinates of the image. For example, the x coordinate value is a coordinate value in the horizontal (scan) direction, and the y coordinate value is a coordinate value in the vertical (scan) direction. A constant value may be set in advance as the size or the number of local areas, or the user may set an arbitrary value as the size or the number of local areas via the external I/F section 500.

The block matching section 324a calculates the motion vector of each local area using a known block matching process, for example. The block matching section 324a outputs the average value of the motion vector calculated corresponding to each local area to the image extraction section 350a as an inter-channel motion vector (Vec_Bx, Vec_By) of the B image.

For example, the block matching process may be implemented by a method that searches the position of a block within the target image that has a high correlation with an arbitrary block within a reference image. In this case, the inter-block relative shift amount corresponds to the motion vector of the block. In one embodiment of the invention, the B image corresponds to the reference image, and the G image corresponds to the block matching target image.

A block having a high correlation may be searched by the block matching process using the sum of squared difference (SSD) or the sum of absolute difference (SAD), for example. Specifically, a block area within the reference image is referred to as I, a block area within the target image is referred to as I′, and the position of the block area I′ having a high correlation with the block area I is calculated. When the pixel position in the block area I and the pixel position in the block area I′ are respectively referred to as pεI and qεI′, and the signal values of the pixels are respectively referred to as Lp and Lq, SSD and SAD are respectively given by the following expressions (2) and (3). It is determined that the correlation is high when the value given by the expression (2) or (3) is small.

$$\begin{matrix}S S D ( I , {I}^{′} ) = {\sum }_{p ∈ I , q ∈ {I}^{′}} {( Lp - Lq )}^{2} & ( 2 ) \\ S A D ( I , {I}^{′} ) = {\sum }_{p ∈ I , q ∈ {I}^{′}} \vert \vert Lp - Lq \vert \vert & ( 3 )\end{matrix}$$

Note that p and q have a two-dimensional value, I and I′ have a two-dimensional area, pεI indicates that the coordinate value p is included in the area I, and “∥m∥” indicates a process that acquires the absolute value of a real number m.

The motion vector interpolation section 325 estimates the inter-channel motion vector (Vec_Rx, Vec_Ry) of the R image based on the inter-channel motion vector (Vec_Bx, Vec_By) of the B image output from the block matching section 324a, and outputs the inter-channel motion vector (Vec_Rx, Vec_Ry) to the image extraction section 350a.

The process performed by the motion vector interpolation section 325 is described in detail below with reference to FIG. 10. The process performed by the motion vector interpolation section 325 differs depending on the time. The process performed at the times t, t+1, and t+2 illustrated in FIG. 10 is described below as an example.

As illustrated in FIG. 10, the RGB image output from the image generation section 310 is acquired at the time t in order of the R image (Rt−2), the B image (Bt−1), and the G image (Gt). Therefore, the inter-channel motion vector (Vec_Rx, Vec_Ry) of the R image is estimated by the following expression (4), for example.

Vec_{—}Rx=2×Vec_{—}Bx

Vec_{—}Ry=2×Vec_{—}By (4)

The RGB image output from the image generation section 310 is acquired at the time t+1 in order of the B image (Bt−1), the G image (Gt), and the R image (Rt+1). Therefore, the inter-channel motion vector (Vec_Rx, Vec_Ry) of the R image is estimated by the following expression (5), for example.

Vec_{—}Rx=Vec_{—}Bx

Vec_{—}Ry=Vec_{—}By (5)

The RGB image output from the image generation section 310 is acquired at the time t+2 in order of the G image (Gt), the R image (Rt+1), and the B image (Bt+2). Therefore, the inter-channel motion vector (Vec_Rx, Vec_Ry) of the R image is estimated by the following expression (6), for example.

Vec_{—}Rx=(Vec_{—}Bx)/2

Vec_{—}Ry=(Vec_{—}By)/2 (6)

The inter-channel motion vector detection section 320 outputs the inter-channel motion vector (Vec_Bx, Vec_By) of the B image and the inter-channel motion vector (Vec_Rx, Vec_Ry) of the R image to the image extraction section 350a.

### 5. Inter-Frame Motion Vector Detection Section

FIG. 14 illustrates a detailed configuration example of the inter-frame motion vector detection section 330a. The inter-frame motion vector detection section 330a includes a G image selection section 321b, the frame memory 331, a gain multiplication section 323b, and the block matching section 324a. The block matching section 324a is connected to the control section 380.

The inter-frame motion vector detection section 330a calculates the inter-frame motion vector of the G image included in the RGB image output from the image generation section 310. The following description is given taking an example of calculating the inter-frame motion vector of the G image included in the RGB image acquired at the time t illustrated in FIG. 11.

The G image selection section 321b selects the G image Gt from the RGB image output from the image generation section 310. The G image selection section 321b then extracts the G image stored in the frame memory 331. The G image Gt−3 acquired at the time t−1 has been stored in the frame memory 331 (described later). The G image selection section 321b outputs the G images Gt and Gt−3 to the gain multiplication section 323b. The G image selection section 321b then resets the information stored in the frame memory 331, and outputs the G image Gt to the frame memory 331. Specifically, the G image Gt stored in the frame memory 331 is handled as the image in the preceding frame at the time t+1.

The gain multiplication section 323a multiplies each pixel of the G image Gt by a gain so that the average signal value of the G image Gt is equal to the average signal value of the G image Gt−3. The gain may be calculated using the expression (1), for example.

The block matching section 324a performs the block matching process on the G image Gt and the G image Gt−3. The block matching process is similar to the block matching process performed by the block matching section 324a included in the inter-channel motion vector detection section 320. Therefore, description thereof is appropriately omitted. The G image Gt corresponds to the reference image, and the G image Gt−3 corresponds to the target image. The block matching section 324a outputs the calculated motion vector to the image extraction section 350a as the inter-frame motion vector (Vec_Gx, Vec_Gy) of the G image. Since the G image is not updated at the times t+1 and t+2 (see FIG. 11), the inter-frame motion vector of the G image is zero.

### 6. Magnification Calculation Section

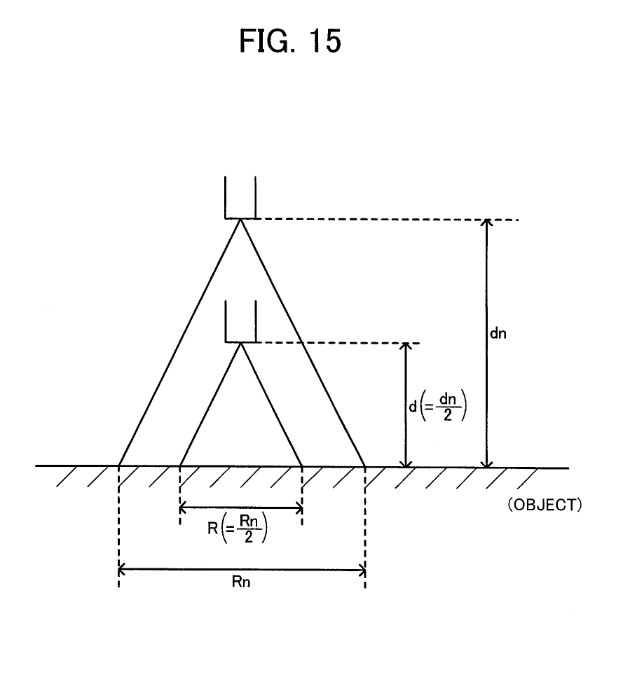

The details of the magnification calculation section 340a are described below with reference to FIG. 15. The magnification calculation section 340a calculates a magnification Z based on the information about the in-focus object plane position d that has been set by the user and output from the control section 380, and the information about the in-focus object plane position dn during normal observation, and outputs the magnification Z to the image extraction section 350a.

The in-focus object plane position of the imaging section 200 used in one embodiment of the invention can be controlled within the range of dmin to dmax (mm). The in-focus object plane position dn during normal observation is dmax (mm). As illustrated in FIG. 15, the size of the imaging area is Rn when the object is captured at the distance dn from the object. The angle of view of the imaging section 200 is constant independently of the in-focus object plane position. In this case, when the in-focus object plane position d is changed to dn/2 (mm), the object is brought into focus when the distance between the end of the imaging section 200 and the object is set to half of that during normal observation. In this case, since the size R of the imaging area is Rn/2, the magnification is twice that during normal observation.

Specifically, the magnification Z is calculated by the following expression (7) using the in-focus object plane position d set by the user and the in-focus object plane position do during normal observation. In one embodiment of the invention, the magnification Z is set to a value within the range of 1 to (dmax/dmin).

$$Z = \frac{d n}{d} \qquad ( 7 )$$

### 7. Image Extraction Section

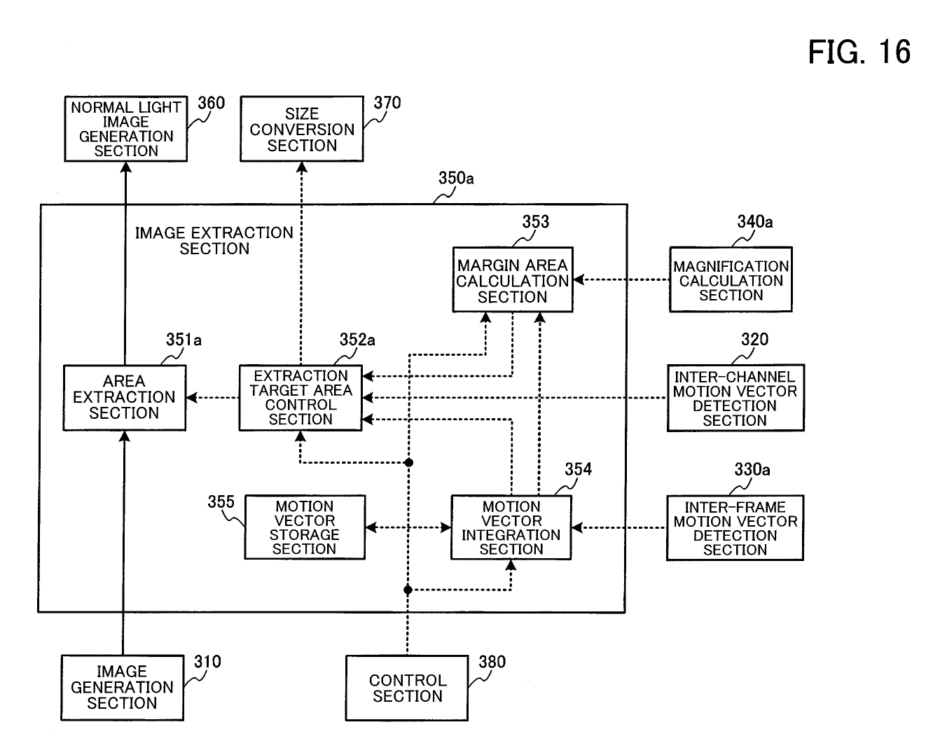

FIG. 16 illustrates a detailed configuration example of the image extraction section 350a. The image extraction section 350a includes an area extraction section 351a, an extraction target area control section 352a, a margin area calculation section 353, a motion vector integration section 354, and a motion vector storage section 355. The margin area calculation section 353 and the motion vector integration section 354 are connected to the control section 380.

The image extraction section 350a determines the size and the coordinates of the area extracted from the RGB image output from the image generation section 310 based on the information output from the inter-channel motion vector detection section 320, the inter-frame motion vector detection section 330a, and the magnification calculation section 340a, and extracts the R′G′B′ image. The image extraction section 350a outputs the extracted R′G′B′ image to the normal light image generation section 360.

More specifically, the motion vector integration section 354 calculates the integrated value (Sum_Gx, Sum_Gy) of the motion vector and the average value Ave_Gr of the absolute value of the motion vector using the inter-frame motion vector (Vec_Gx, Vec_Gy) of the G image output from the inter-frame motion vector detection section 330a, and the motion vector stored in the motion vector storage section 355. Note that the process performed at a given time t is described below as an example.

The motion vector storage section 355 stores the integrated value (Sum_Gx_M, Sum_Gy_M) of the inter-frame motion vector of the G image from the initial frame to the time t−1, the integrated value (Abs_Gx_M, Abs_Gy_M) of the absolute value of the inter-frame motion vector of the G image, and information about a motion vector integration count T_M. The integrated value (Sum_Gx, Sum_Gy) of the motion vector at the time t is calculated by the following expression (8). The motion vector integration count T at the time t is calculated by the following expression (9). The average value Ave_Gr of the absolute value of the motion vector at the time t is calculated by the following expression (10).

Sum_{—}Gx=Sum_{—}Gx_{—}M+Vec_{—}Gx

Sum_{—}Gy=Sum_{—}Gy_{—}M+Vec_{—}Gy (8)

T=T_{—}M+1 (9)

Ave_{—}Gr=(Abs_{—}Gx+Abs_{—}Gy)/T (10)

Abs_Gx and Abs_Gy in the expression (10) are integrated values of the absolute value of the motion vector, and calculated using the following expression (11).

Abs_{—}Gx=Abs_{—}Gx_{—}M+∥Vec_{—}Gx∥

Abs_{—}Gy=Abs_{—}Gy_{—}M+∥Vec_{—}Gy∥ (11)

The motion vector integration section 354 outputs the integrated value (Sum_Gx, Sum_Gy) of the motion vector calculated using the expression (8) to the extraction target area control section 352a, and outputs the average value Ave_Gr of the absolute value of the motion vector calculated using the expression (10) to the margin area calculation section 353. The motion vector integration section 354 outputs the integrated value (Sum_Gx, Sum_Gy) of the motion vector, the integrated value (Abs_Gx, Abs_Gy) of the absolute value of the motion vector calculated using the expression (11), and the motion vector integration count T to the motion vector storage section 355. Note that the motion vector integration section 354 resets the information stored in the motion vector storage section 355 when outputting the above values.

The margin area calculation section 353 calculates the size of the margin area used when extracting the R′G′B′ image from the RGB image based on the information about the magnification Z output from the magnification calculation section 340a. More specifically, the margin area calculation section 353 calculates the size Space_X and Space_Y of the margin area in the x and y directions using the following expression (12).

Space_{—}X=Z×Space_{—}Xₘᵢₙ

Space_{—}Y=Z×Space_{—}Yₘᵢₙ (12)

where, Space_Xₘᵢₙ and Space_Yₘᵢₙ are the size of the margin area during normal observation. A constant value may be set as Space_Xₘᵢₙ and Space_Yₘᵢₙ in advance, or the user may set an arbitrary value as Space_Xₘᵢₙ and Space_Yₘᵢₙ via the external I/F section 500.

For example, a margin area having a size 10 times that during normal observation at a magnification Z of 1 is set during magnifying observation at a magnification Z of 10. Specifically, since the margin area is set in proportion to the magnification, it is possible to implement stable shake canceling even during magnifying observation.

The margin area calculation section 353 refers to the average value Ave_Gr of the absolute value of the motion vector output from the motion vector integration section 354, and updates the size of the margin area calculated by the expression (12) using the following expression (13) when the average value Ave_Gr is larger than a threshold value Vmax.

Space_{—}X=Coₘₐₓ×Space_{—}X

Space_{—}Y=Coₘₐₓ×Space_{—}Y (13)

The margin area calculation section 353 updates the size of the margin area calculated by the expression (12) using the following expression (14) when the average value Ave_Gr of the absolute value of the motion vector is smaller than a threshold value Vmin.

Space_{—}X=Coₘᵢₙ×Space_{—}X

Space_{—}Y=Coₘᵢₙ×Space_{—}Y (14)

where, Coₘₐₓ is an arbitrary real number that is larger than 1, and Coₘᵢₙ is an arbitrary real number that is smaller than 1. Specifically, when the average value Ave_Gr of the motion vector is larger than the threshold value Vmax, the size of the margin area is updated with a larger value using the expression (13). When the average value Ave_Gr of the motion vector is smaller than the threshold value Vmin, the size of the margin area is updated with a smaller value using the expression (14).

Note that a constant value may be set as the threshold values Vmax and Vmin and the coefficients Coₘₐₓ and Coₘᵢₙ in advance, or the user may set an arbitrary value as the threshold values Vmax and Vmin and the coefficients Coₘₐₓ and Coₘᵢₙ via the external I/F section 500.

The above process makes it possible to control the margin area corresponding to the average value Ave_Gr of the absolute value of the motion vector. This makes it possible to implement appropriate shake canceling corresponding to the amount of shake.

For example, when the scope is positioned in a gullet, the position of the esophageal mucous membrane (i.e., object) changes to a large extent due to pulsation of the heart. In this case, it is considered that the effects of the shake canceling process cannot be sufficiently obtained due to an increase in inter-frame motion vector. Therefore, the size of the margin area is increased when the average value Ave_Gr of the absolute value of the motion vector is larger than the threshold value Vmax (see the expression (13)), so that stable shake canceling can be implemented.

The object may be observed during magnifying observation in a state in which a hood is attached to the end of the imaging section 200, and comes in close contact with the object in order to reduce the effects of shake. In this case, the effects of shake are reduced since the positional relationship between the imaging section 200 and the object is fixed. In this case, the average value Ave_Gr of the absolute value of the motion vector decreases. Therefore, the size of the area used for display can be increased by reducing the size of the margin area (see the expression (14)). This makes it possible to present a display image that captures a wider area to the user.

The extraction target area control section 352a then determines the conditions employed when extracting the R′G′B′ image from the RGB image output from the image generation section 310 based on the information about the margin area output from the margin area calculation section 353, the integrated value of the motion vector output from the motion vector integration section 354, and the inter-channel motion vector output from the inter-channel motion vector detection section 320. More specifically, the extraction target area control section 352a determines the starting point coordinates when extracting the R′G′B′ image, and the numbers imx and imy of pixels of the R′G′B′ image in the x and y directions, as the conditions employed when extracting the R′G′B′ image.

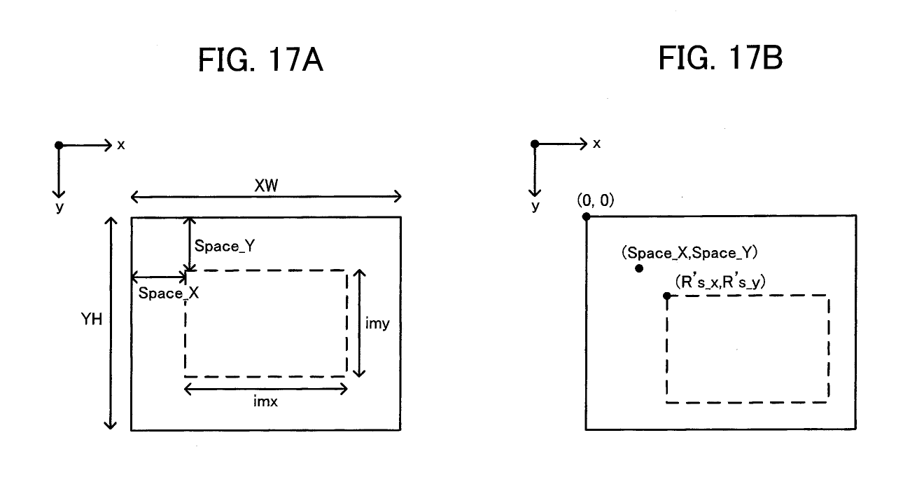

The extraction target area control section 352a calculates the numbers imx and imy of pixels of the R′G′B′ image using the following expression (15). Note that XW is the number of pixels of the image acquired by the imaging section 200 in the x direction, and XY is the number of pixels of the image acquired by the imaging section 200 in the y direction (see FIG. 17A).

imx=XW−2×Space_{—}X

imy=YH−2×Space_{—}Y (15)

The extraction target area control section 352a calculates the starting point coordinates using the following expression (16). Since the starting point coordinates differ between the R′ image, the G′ image, and the B′ image, the starting point coordinates are calculated for each of the R′ image, the G′ image, and the B′ image (see the expression (16)). Note that R's_x and R's_y are the starting point coordinate values of the R′ image acquired by the imaging section 200 (see FIG. 17B). G's_x and G's_y are the starting point coordinate values of the G′ image, and B's_x and B's_y are the starting point coordinate values of the B′ image.

R's_{—}x=Space_{—}X−Sum_{—}Gx−Vec_{—}Rx

R's_{—}y=Space_{—}Y−Sum_{—}Gy−Vec_{—}Ry

G's_{—}x=Space_{—}X−Sum_{—}Gx

G's_{—}y=Space_{—}Y−Sum_{—}Gy

B's_{—}x=Space_{—}X−Sum_{—}Gx−Vec_{—}Bx

B's_{—}y=Space_{—}Y−Sum_{—}Gy−Vec_{—}By (16)

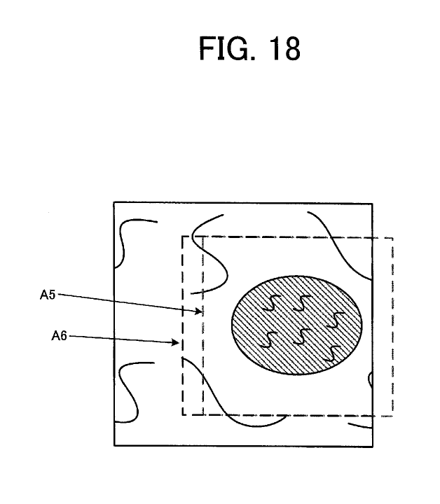

The extraction target area control section 352a performs a clipping process (see the following expression (17)) on the starting point coordinates calculated using the expression (16). The clipping process corresponds to a process that shifts the starting point coordinates when part of the extraction target area is positioned outside the captured image (see A5 in FIG. 18). The extraction target area is positioned within the captured image as a result of performing the clipping process (see A6). Note that the clipping process is similarly performed on the G′ image and the B′ image.

$$\begin{matrix}{R}^{′} s\_x = & 0 & if ( {R}^{′} s\_x < 0 ) \\ & XW - imx & else if ( {R}^{′} s\_x > XW - imx ) \\ & {R}^{′} s\_x & else \\ {R}^{′} s\_y = & 0 & if ( {R}^{′} s\_y < 0 ) \\ & YH - imy & else if ( {R}^{′} s\_y > YH - imy ) \\ & {R}^{′} s\_y & else\end{matrix} \} \qquad ( 17 )$$

The extraction target area control section 352a outputs the starting point coordinates after the clipping process and the number of pixels of the R′G′B′ image to the area extraction section 351a. The extraction target area control section 352a also outputs the ratios zoom_x and zoom_y of the number of pixels of the RGB image to the number of pixels of the R′G′B′ image, to the size conversion section 370. The ratios zoom_x and zoom_y are calculated using the following expression (18).

zoom_{—}x=XW/imx

zoom_{—}y=YH/imy (18)

The area extraction section 351a extracts the R′G′B′ image from the RGB image using the information about the starting point coordinates and the number of pixels of the R′G′B′ image output from the extraction target area control section 352a, and outputs the R′G′B′ image to the normal light image generation section 360.

The above process makes it possible to implement stable shake canceling during magnifying observation using an optical system of which the in-focus object plane position is changed. This makes it possible to suppress or prevent a situation in which the attention area is positioned outside the field of view, and is missed during magnifying observation due to an incapability to implement shake canceling.

Note that the shake canceling process may be turned ON/OFF via the external I/F section 500. For example, when the shake canceling function has been set to “OFF” using the external I/F section 500, the information is transmitted to the control section 380. The control section 380 outputs a trigger signal that indicates that the shake canceling process has been set to “OFF” to the motion vector integration section 354 and the margin area calculation section 353. Note that the trigger signal is continuously output during a period in which the shake canceling process is set to “OFF”.

When the control section 380 has output the trigger signal that indicates that the shake canceling process has been set to “OFF”, the motion vector integration section 354 sets the information about the motion vector and the integration count stored in the motion vector storage section 355 to “0”. The information about the integrated value (Sum_Gx, Sum_Gy) of the motion vector and the average value Ave_Gr of the absolute value of the motion vector output from the motion vector integration section 354 is set to “0” during a period in which the trigger signal that indicates that the shake canceling process has been set to “OFF” is output. The integration count (see the expression (9)) is also not counted. When the control section 380 has output the trigger signal that indicates that the shake canceling process has been set to “OFF”, the values Space_X and Space_Y of the margin area output from the margin area calculation section 353 are set to “0”.

When performing magnifying observation using a magnifying endoscope, the observation state is significantly affected by the relative motion of the imaging section provided at the end of the endoscope with respect to tissue that is the observation target. Specifically, a large amount of shake is observed on the monitor of the endoscope even if the motion is small, so that diagnosis is hindered.

Moreover, the attention area (e.g., lesion area) may be missed due to the effects of shake. Since the field of view is very narrow during magnifying observation, it is difficult to find the missing attention area. Therefore, the doctor must search the missing attention area in a state in which the observation state is switched from magnifying observation to normal observation to increase the field of view, and then observe the attention area after switching the observation state from normal observation to magnifying observation. It is troublesome for the doctor to repeat such an operation, and such an operation increases the diagnosis time.

JP-A-3-16470 discloses a method that implements shake canceling using a motion vector calculated from a plurality of images captured in time series. However, since the method disclosed in JP-A-3-16470 does not take account of the effects of an increase in shake due to the magnification, the shake canceling process does not sufficiently function when the magnification is high.

Since the amount of shake tends to increase in proportion to the magnification during magnifying observation (diagnosis) using an endoscope, it becomes difficult to implement shake canceling as the magnification increases. Moreover, it is likely that the observation target is missed if shake canceling cannot be implemented, and it is necessary to repeat the operation that increases the magnification from a low magnification when the observation target has been missed.

According to one embodiment of the invention, an image processing device (image processing section 300) that processes an image acquired by the imaging section 200 that enables magnifying observation, includes a motion information acquisition section (inter-channel motion vector detection section 320 and inter-frame motion vector detection section 330a), an imaging magnification calculation section (magnification calculation section 340a), and the image extraction section 350a (see FIG. 5).

The motion information acquisition section acquires motion information that indicates the relative motion of the imaging section 200 with respect to the object. The imaging magnification calculation section calculates the imaging magnification Z (magnification) of the imaging section 200. The image extraction section 350a extracts an image within a specific area from the captured image acquired by the imaging section 200 as an extracted image. The image extraction section 350a sets the position (e.g., R's_x and R's_y) of the specific area within the captured image based on the motion information (see FIG. 17B). The image extraction section 350a sets the size (Space_X and Space_Y) of the margin area based on the imaging magnification Z, the margin area being an area except the specific area in the captured image (see FIG. 17A).

It is possible to change the margin when setting the position of the specific area within the captured image corresponding to the magnification by thus setting the margin area corresponding to the magnification. This makes it possible to improve the shake followability during magnifying observation, and implement stable shake canceling. It is possible to reduce the possibility that the observation target is missed by thus improving the shake followability.

Note that the term “motion information” used herein refers to information that indicates the relative motion of the imaging section 200 with respect to the object between different timings. For example, the motion information is information that indicates the relative position, the moving distance, the speed, the motion vector, or the like. In one embodiment of the invention, the inter-channel motion vector between the G image and the B image is acquired as the motion information between the capture timing of the G image and the capture timing of the B image, for example. Note that the motion information is not limited to motion information calculated from images, but may be motion information (e.g., moving distance or speed) obtained by sensing the motion using a motion sensor or the like.

The image extraction section 350a may set the size Space_X and Space_Y of the margin area to the size that is proportional to the imaging magnification Z (see the expression (12)). More specifically, the image extraction section 350a may set the size Space_X and Space_Y of the margin area to the size obtained by multiplying the reference size Space_Xₘᵢₙ and Space_Yₘᵢₙ by the imaging magnification Z, the reference size being a reference of the size of the margin area.

According to the above configuration, since the size of the margin area can be increased as the magnification increases, it is possible to follow a larger amount of shake as the magnification increases. This makes it possible to cancel a large amount of shake during magnifying observation as compared with the comparative example in which the size of the margin area is constant (see FIG. 4, for example).

Although an example in which the size Space_X and Space_Y of the margin area is linearly proportional to the imaging magnification Z has been described above, another configuration may also be employed. For example, the size Space_X and Space_Y of the margin area may non-linearly increase as the imaging magnification Z increases.

The image extraction section 350a may update the size Space_X and Space_Y of the margin area that has been set to the size obtained by multiplying the reference size Space_Xₘᵢₙ and Space_Yₘᵢₙ by the imaging magnification Z based on the motion information (see the expressions (13) and (14)). The term “update” used herein refers to resetting a variable to a new value. For example, the margin area calculation section 353 illustrated in FIG. 16 includes a storage section that is not illustrated in FIG. 16, and overwrites the value stored in a storage area of the storage section that stores the size of the margin area with a new value.

This makes it possible to adjust the size Space_X and Space_Y of the margin area corresponding to the relative motion of the imaging section 200 with respect to the object. Therefore, it is possible to improve the shake followability by increasing the size of the margin area when the amount of shake has increased.

Specifically, the image extraction section 350a may update the size Space_X and Space_Y of the margin area based on the average value Ave_Gr of the motion information within a given period (e.g., a period corresponding to the integration count T (see the expression (9)).

More specifically, the image extraction section 350a may update the size of the margin area with the size Coₘₐₓ×Space_X and Coₘᵢₙ×Space_Y that is larger than the size set based on the imaging magnification Z when the average value Ave_Gr of the motion information is larger than the first threshold value Vmax. The image extraction section 350a may update the size of the margin area with the size Coₘᵢₙ×Space_X and Coₘᵢₙ×Space_Y that is smaller than the size set based on the imaging magnification Z when the average value Ave_Gr of the motion information is smaller than the second threshold value Vmin

This makes it possible to adjust the size of the margin area corresponding to the amount of shake. Specifically, it is possible to increase the amount of shake that can be canceled by increasing the size of the margin area when the amount of shake is larger than the threshold value. On the other hand, it is possible to increase the display area, and increase the amount of information presented to the user by decreasing the size of the margin area when the amount of shake is smaller than the threshold value.

The motion information may be the motion vectors Vec_Gx and Vec_Gy that indicate the motion of the object within the captured image. The image extraction section 350a may update the size Space_X and Space_Y of the margin area based on the average value Ave_Gr of the absolute value of the motion vector within a given period (see the expressions (10) and (11)).

It is possible to use the magnitude of the motion vector as the amount of shake, and set the size of the margin area corresponding to the amount of shake by thus utilizing the absolute value of the motion vector.

The image extraction section 350a may reset the specific area within the captured image when it has been determined that at least part of the specific area is positioned outside the captured image (see FIG. 18). More specifically, the image extraction section 350a may reset the position of the specific area within the margin area when the magnitude of the motion vector has exceeded the size of the margin area. For example, when the left end of the specific area is positioned outside the captured image (e.g., when R's_x<0), the image extraction section 350a may set the value R's_x to 0. When the right end of the specific area is positioned outside the captured image (e.g., when R's_x>2×Space_X (=XW−imx)), the image extraction section 350a may set the value R's_x to 2×Space_X.

This makes it possible to perform the clipping process when the extraction target area has been partially positioned outside the captured image. Specifically, it is possible to set the specific area at a position at which the display image can be extracted, and display the image even if the amount of shake has reached a value that cannot be canceled.

The image processing device may include an in-focus object plane position information acquisition section (control section 380) that acquires in-focus object plane position information about the imaging section 200 (condenser lens 230) (see FIG. 5). The imaging magnification Z may be changed by changing the distance between the imaging section 200 and the object (see FIG. 15). In this case, the imaging magnification calculation section may calculate the imaging magnification Z based on the in-focus object plane position information. More specifically, the imaging magnification calculation section may calculate the imaging magnification Z based on the ratio “dn/d” of the reference in-focus object plane position dn to the in-focus object plane position d indicated by the in-focus object plane position information.

This makes it possible to calculate the imaging magnification Z from the in-focus object plane position information about the imaging section 200 when using an endoscope apparatus that magnifies the object by moving the end of the imaging section 200 closer to the object.

The motion information acquisition section may acquire the motion vector (inter-frame motion vector or inter-channel motion vector) that indicates the motion of the object within the captured image based on at least two captured images acquired at different times (t−1, t, t+1, . . . ) (see FIGS. 10 and 11).

More specifically, the imaging section 200 may sequentially acquire a first color signal image, a second color signal image, and a third color signal image (R image, G image, and B image) in time series as the captured image. The motion information acquisition section may acquire the motion vector that indicates the motion of the object between the first color signal image, the second color signal image, and the third color signal image as the inter-channel motion vector (e.g., Vec_Bx and Vec_By). The image extraction section 350a may extract the extracted image from the first color signal image, the second color signal image, and the third color signal image based on the inter-channel motion vector.

This makes it possible to perform the shake canceling process using the motion vector between the captured images as the information about the relative motion of the imaging section 200 with respect to the object. It is also possible to suppress a frame-sequential color shift by canceling inter-channel shake when using a frame-sequential endoscope apparatus.

The image processing device may include the size conversion section 370 that converts the size of the extracted image to a given size (i.e., a given number of pixels (e.g., the same size as that of the captured image)) that can be displayed on the display section 400 when the size of the extracted image changes corresponding to the imaging magnification (see FIG. 5). More specifically, the imaging section 200 may acquire a series of images that is a moving image as the captured image, and the size conversion section 370 may convert a series of extracted images extracted from the series of images to have an identical size.

According to the above configuration, since the extracted image that changes in size corresponding to the magnification can be converted to have a constant size, it is possible to display a display image having a constant size independently of the magnification.

### 8. Second Configuration Example of Endoscope Apparatus

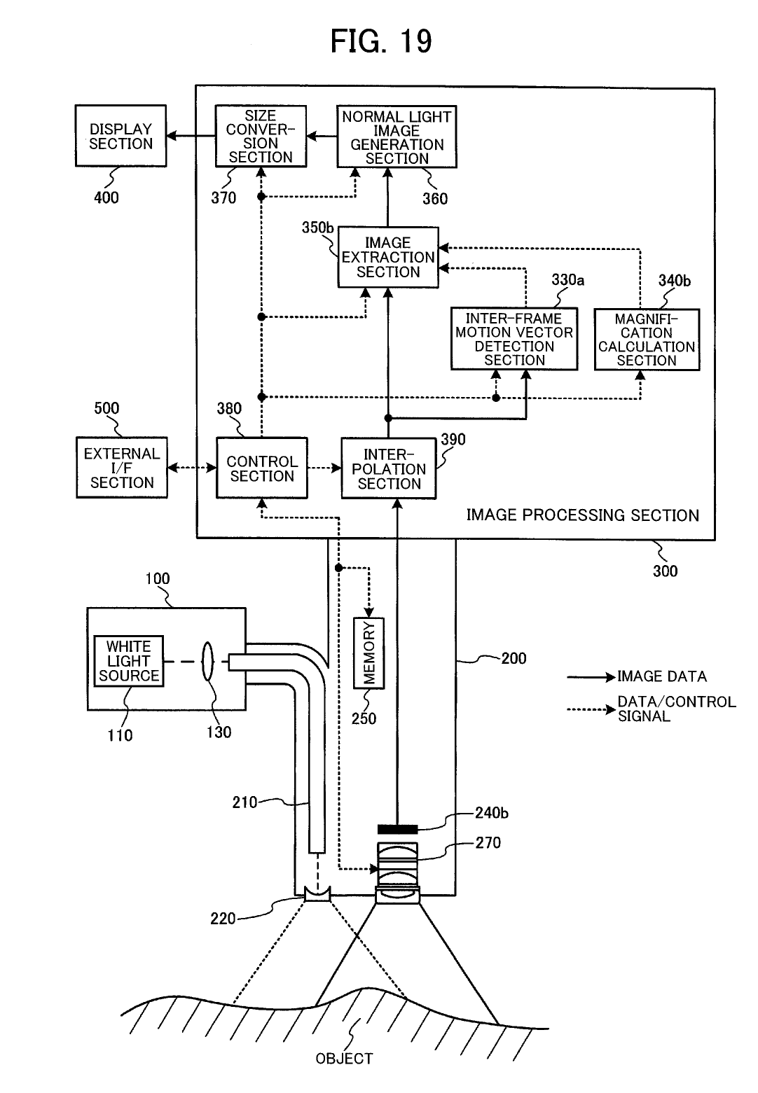

FIG. 19 illustrates a second configuration example of the endoscope apparatus as a configuration example in which the magnification is calculated based on the angle of view of the imaging section 200. The endoscope apparatus includes a light source section 100, an imaging section 200, an image processing section 300, a display section 400, and an external I/F section 500. Note that the same elements as those illustrated in FIG. 5 and the like are respectively indicated by the same reference signs, and description thereof is appropriately omitted.

The light source section 100 includes a white light source 110 that emits white light, and a lens 130 that focuses the white light on a light guide fiber 210.

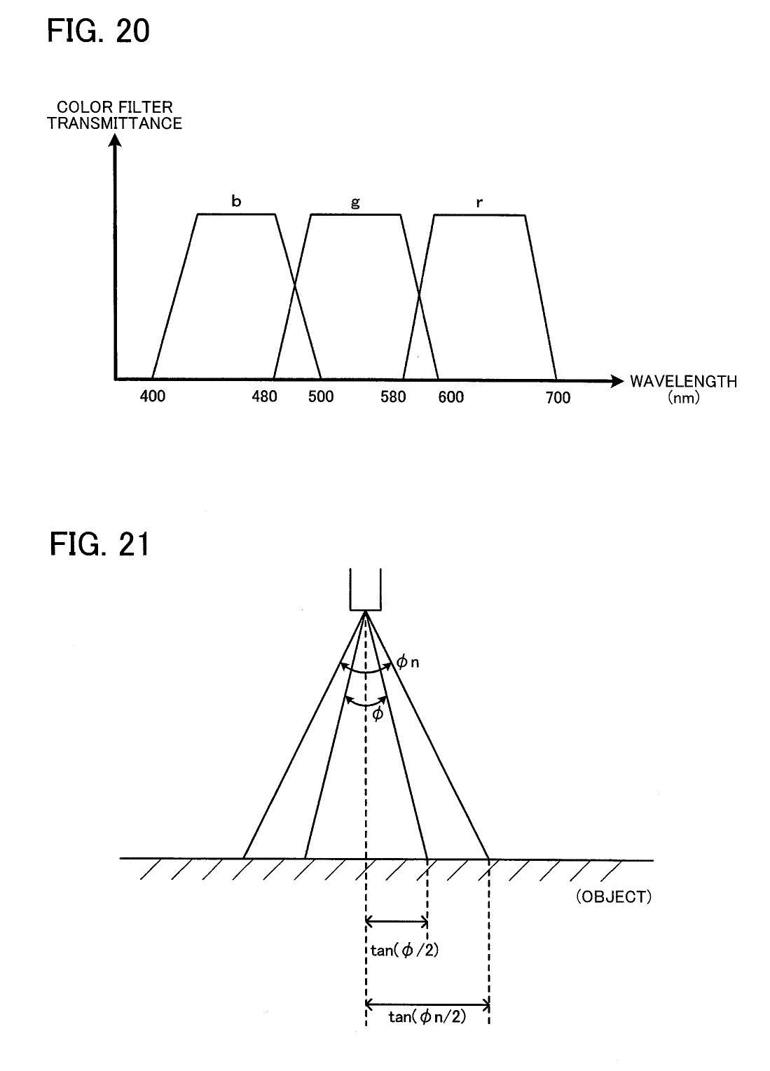

The imaging section 200 includes the light guide fiber 210, an illumination lens 220, a condenser lens 270, an image sensor 240b, and a memory 250. The image sensor 240b includes Bayer-array color filters r, g, and b. As illustrated in FIG. 20, the filter r has spectral characteristics that allow light having a wavelength of 580 to 700 nm to pass through, the filter g has spectral characteristics that allow light having a wavelength of 480 to 600 nm to pass through, and the filter b has spectral characteristics that allow light having a wavelength of 400 to 500 nm to pass through.

The angle of view of the condenser lens 270 can be variably controlled. For example, the angle of view of the condenser lens 270 can be adjusted within the range of φmin to φmax (°). The angle of view φn during normal observation is φmax (°). The user can set an arbitrary angle of view via the external I/F section 500. The information about the angle of view φ set by the user via the external VP section 500 is transmitted to a control section 380, and the control section 380 changes the angle of view of the condenser lens 270 by controlling the condenser lens 270 corresponding to the information about the angle of view φ.

The angle of view control range φmin to φmax (°) differs depending on the connected scope. The control section 380 can identify the type of the connected scope by referring to the identification number of each scope stored in the memory 250, and acquire the information about the angle of view control range φmin to φmax (°) and the angle of view φn during normal observation.

The control section 380 outputs information about the magnification to a magnification calculation section 340b (described later). The information about the magnification includes information about the angle of view φ set by the user, information about the angle of view φn during normal observation, and information about the minimum value φmin of the angle of view.

The image processing section 300 includes an interpolation section 390, an inter-frame motion vector detection section 330a, the magnification calculation section 340b, an image extraction section 350b, a normal light image generation section 360, a size conversion section 370, and a control section 380. The interpolation section 390, the inter-frame motion vector detection section 330a, the magnification calculation section 340b, the image extraction section 350b, the normal light image generation section 360, and the size conversion section 370 are connected to the control section 380. The process performed by the inter-frame motion vector detection section 330a, the process performed by the normal light image generation section 360, and the process performed by the size conversion section 370 are the same as described above (see FIG. 5 and the like). Therefore, description thereof is omitted.

The interpolation section 390 performs an interpolation process on a Bayer image acquired by the image sensor 240b to generate an RGB image. For example, a known bicubic interpolation process may be used as the interpolation process. The interpolation section 390 outputs the generated RGB image to the image extraction section 350b and the inter-frame motion vector detection section 330a.

The magnification calculation section 340b calculates a magnification Z′ using the information about the angle of view φ that has been set by the user and the information about the angle of view φn during normal observation, and outputs the magnification Z′ to the image extraction section 350b. As illustrated in FIG. 21, the ratio of the size of the imaging area at the angle of view φn to the size of the imaging area at the angle of view φ is the magnification Z′ when the distance from the object to the imaging section 200 is identical. Specifically, the magnification Z′ is calculated by the following expression (19). In one embodiment of the invention, the magnification Z′ is set to a value within the range of 1 to (tan(φn/2)/tan(φmin/2)).

$${Z}^{′} = \frac{tan ( φ n / 2 )}{tan ( φ / 2 )} \qquad ( 19 )$$

The image extraction section 350b extracts an image within a specific area (extraction target area) from the RGB image output from the interpolation section 390 as an R′G′B′ image based on the information output from the inter-frame motion vector detection section 330a and the magnification calculation section 340b.

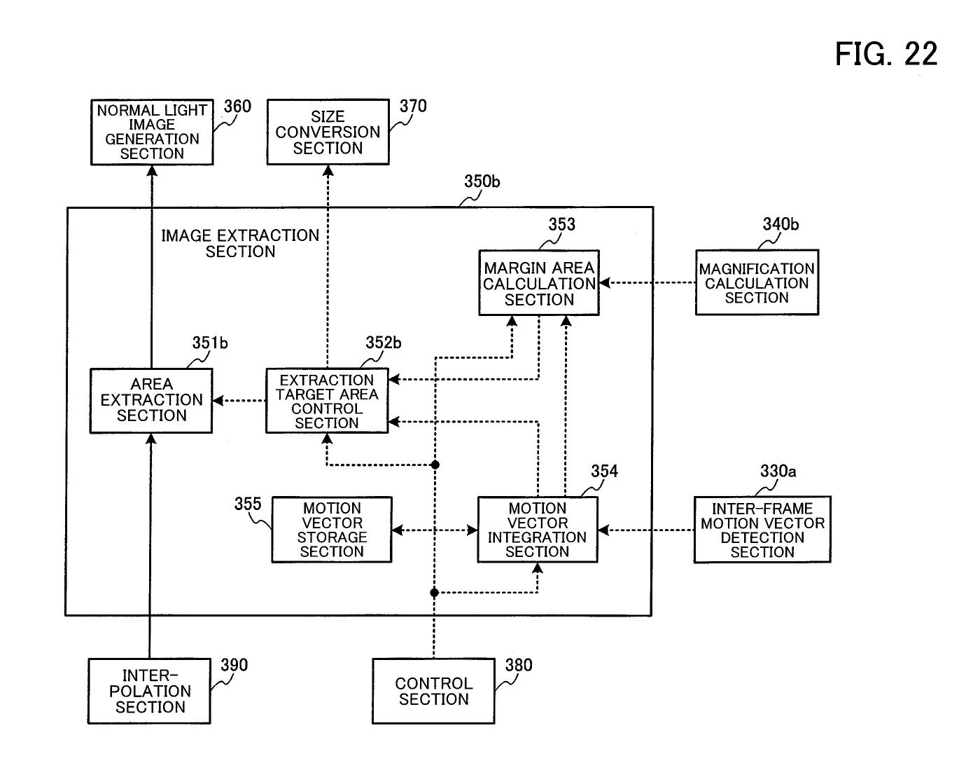

FIG. 22 illustrates a detailed configuration example of the image extraction section 350b. The image extraction section 350b includes an area extraction section 351b, an extraction target area control section 352b, a margin area calculation section 353, a motion vector integration section 354, and a motion vector storage section 355. The margin area calculation section 353 and the motion vector integration section 354 are connected to the control section 380. Note that the process performed by the margin area calculation section 353, the process performed by the motion vector integration section 354, and the process performed by the motion vector storage section 355 are the same as described above (see FIG. 16 and the like). Therefore, description thereof is omitted.

The extraction target area control section 352b determines the starting point coordinates and the numbers imx and imy of pixels when extracting the R′G′B′ image from the RGB image based on information about the size Space_X and Space_Y of the margin area and the integrated value (Sum_Gx, Sum_Gy) of the motion vector. The numbers imx and imy of pixels of the R′G′B′ image are calculated using the expression (15) (see FIG. 16 and the like).

The starting point coordinates are calculated using the following expression (20).

In the second configuration example, a color shift does not occur since the R image, the G image, and the B image are acquired at the same time. Therefore, it is unnecessary to calculate the starting point coordinates corresponding to each of the R′ image, the G′ image, and the B′ image, and only one set of starting point coordinates (I's_x, I's_y) is calculated.

I's_{—}x=Space_{—}X−Sum_{—}Gx

I's_{—}y=Space_{—}Y−Sum_{—}Gy (20)

A clipping process is performed using the expression (17) on the starting point coordinates (I's_x, I's_y) calculated using the expression (20). The extraction target area control section 352b calculates the ratios zoom_x and zoom_y of the number of pixels of the RGB image to the number of pixels of the R′G′B′ image using the expression (18), and outputs the ratios zoom_x and zoom_y to the size conversion section 370.

The above process makes it possible to implement stable shake canceling even during magnifying observation using an optical system of which the angle of view is changed. Moreover, it is possible to suppress a situation in which the attention area is missed during magnifying observation. In the second configuration example, the R image, the G image, and the B image are acquired at the same time. This makes it possible to simplify the process since it is unnecessary to take account of a color shift (see FIG. 5 and the like). Moreover, since it is unnecessary to calculate the inter-channel motion vector, the capacity of the frame memory can be reduced.

According to the second configuration example, the image processing device may include an angle-of-view information acquisition section (control section 380) that acquires angle-of-view information about the imaging section 200 (see FIG. 19). The imaging magnification calculation section (magnification calculation section 340b) may calculate the imaging magnification Z′ based on the angle-of-view information. More specifically, the imaging magnification calculation section may calculate the imaging magnification Z based on the ratio of tan(φn/2) to tan(φ/2) when the reference angle of view is φn, and the angle of view indicated by the angle-of-view information is φ (see FIG. 21).

This makes it possible to calculate the imaging magnification Z′ of the imaging section 200 from the angle-of-view information when the endoscope apparatus is configured to implement magnifying observation of the object using a zoom function (e.g., optical zoom function) of the imaging section 200.

### 9. Third Configuration Example of Endoscope Apparatus

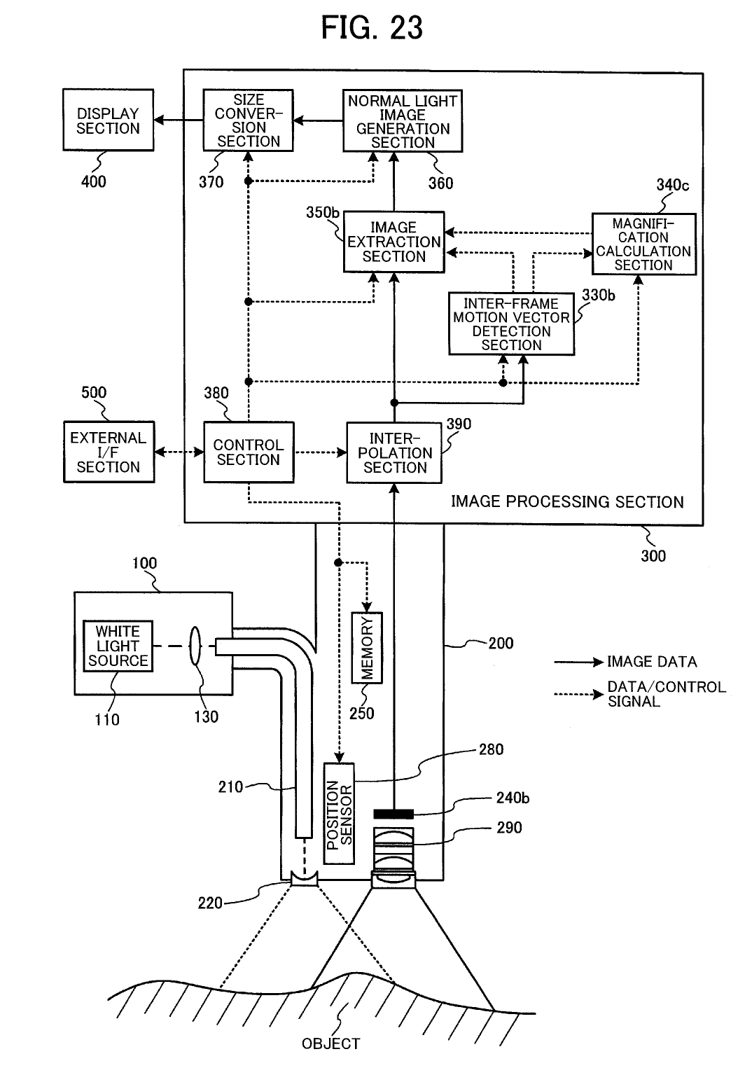

FIG. 23 illustrates a third configuration example of the endoscope apparatus as a configuration example in which the magnification is calculated based on information from a position sensor and a motion vector. The endoscope apparatus includes a light source section 100, an imaging section 200, an image processing section 300, a display section 400, and an external I/F section 500. Note that the same elements as those illustrated in FIG. 5 and the like are respectively indicated by the same reference signs, and description thereof is appropriately omitted.

The imaging section 200 includes the light guide fiber 210, an illumination lens 220, a condenser lens 290, an image sensor 240b, a memory 250, and a position sensor 280. Note that the image sensor 240b and the memory 250 are the same as those illustrated in FIG. 19 and the like.

The position sensor 280 detects the moving amount of the end of the imaging section. For example, the position sensor 280 is implemented by an acceleration sensor that senses the translation amount in three directions, and outputs the moving amount of the end of the imaging section that translates relative to the surface of the object. The moving amount is the moving distance of the end of the imaging section, or the motion vector, for example. The position sensor 280 is connected to the control section 380, and information about the moving amount detected by the position sensor 280 is output to the control section 380. Note that the position sensor 280 may includes a triaxial gyrosensor, and may output the rotation angle of the end of the imaging section as the moving amount.

The image processing section 300 includes an interpolation section 390, an inter-frame motion vector detection section 330b, a magnification calculation section 340c, an image extraction section 350b, a normal light image generation section 360, a size conversion section 370, and a control section 380. The process performed by the image extraction section 350b, the process performed by the normal light image generation section 360, the process performed by the size conversion section 370, and the process performed by the interpolation section 390 are the same as described above (see FIG. 19 and the like). Therefore, description thereof is omitted.

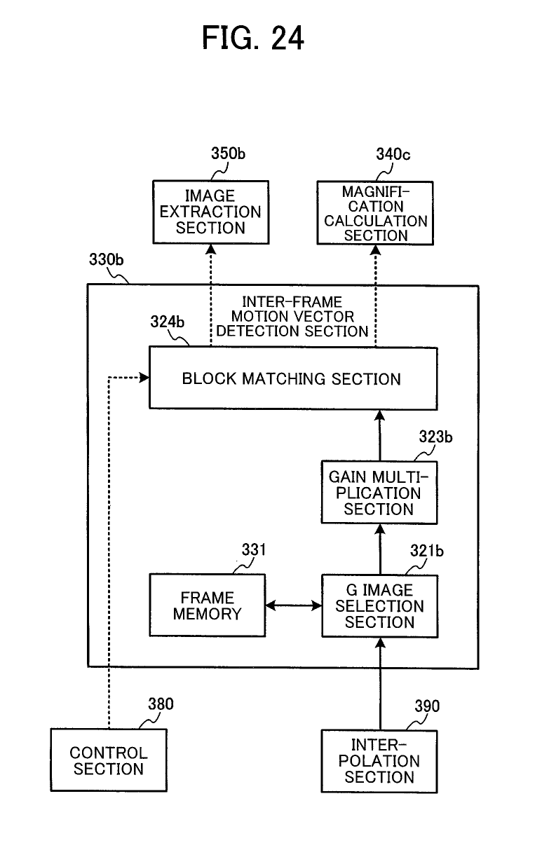

FIG. 24 illustrates a detailed configuration example of the inter-frame motion vector detection section 330b. The inter-frame motion vector detection section 330b includes a G image selection section 321b, a frame memory 331, a gain multiplication section 323b, and a block matching section 324b. The process performed by the G image selection section 321b, the process performed by the frame memory 331, and the process performed by the gain multiplication section 323b are the same as described above (see FIG. 5 and the like).

The block matching section 324b detects the motion vector of each local area, and outputs the average value of the motion vector of each local area to the image extraction section 350b (see FIG. 13 and the like). The block matching section 324b also outputs information about the coordinates and the motion vector of each local area to the magnification calculation section 340c. The coordinates of each local area may be the center coordinates of each local area, for example.

The magnification calculation section 340c is described below. When performing magnifying observation (diagnosis) using an endoscope, the magnification is normally increased by moving the scope closer to tissue (object). In this case, the magnification can be calculated if the distance between the end of the imaging section 200 and the object can be determined.

The magnification calculation section 340c estimates the average distance between the end of the imaging section 200 and the object based on the information about the moving amount (e.g., motion vector) of the end of the imaging section 200 output from the control section 380, and the information about the coordinates and the motion vector of each local area output from the inter-frame motion vector detection section 330b, and calculates the magnification.

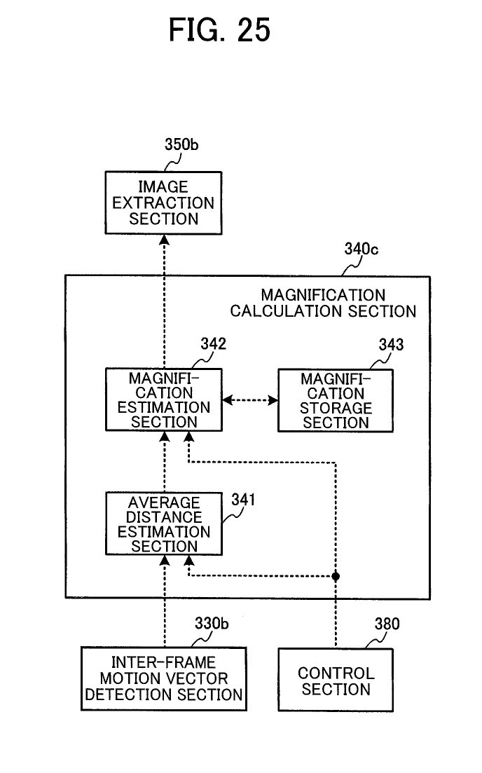

FIG. 25 illustrates a detailed configuration example of the magnification calculation section 340c. The magnification calculation section 340c includes an average distance estimation section 341, a magnification estimation section 342, and a magnification storage section 343.

The average distance estimation section 341 estimates the average distance between the end of the imaging section 200 and the object based on the information about the moving amount of the end of the imaging section 200 output from the control section 380, and the information about the coordinates and the motion vector of each local area output from the inter-frame motion vector detection section 330b. The following description is given taking an example in which the average distance is calculated at a given time t.

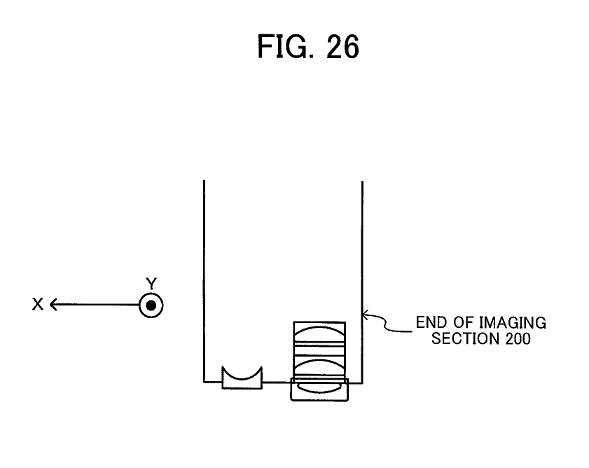

The inter-frame motion vector detection section 330b outputs the motion vector and the coordinates of each local area calculated from the images acquired at the time t and a time t−1 that precedes the time t by one frame. The control section 380 outputs the moving amount of the end of the imaging section 200 between the time t and the time t−1. More specifically, the X-axis moving amount and the Y-axis moving amount (see FIG. 26) are output as the moving amount.

The information about the moving amount output from the control section 380 is referred to as (TX, TY). The information (TX, TY) indicates the moving amount of the end of the imaging section 200 from the time t−1 to the time t. The relative positional relationship between the end of the imaging section 200 and the object at each time (t and t−1) is determined by the moving amount. The motion vector between the images acquired at the time t and the time t−1 is also acquired.

Since the relative positional relationship between the end of the imaging section 200 and the object at each time, and the relationship between the images acquired at the respective times have been determined, the average distance diff_val between the end of the imaging section 200 and the object can be estimated using a known triangulation principle.

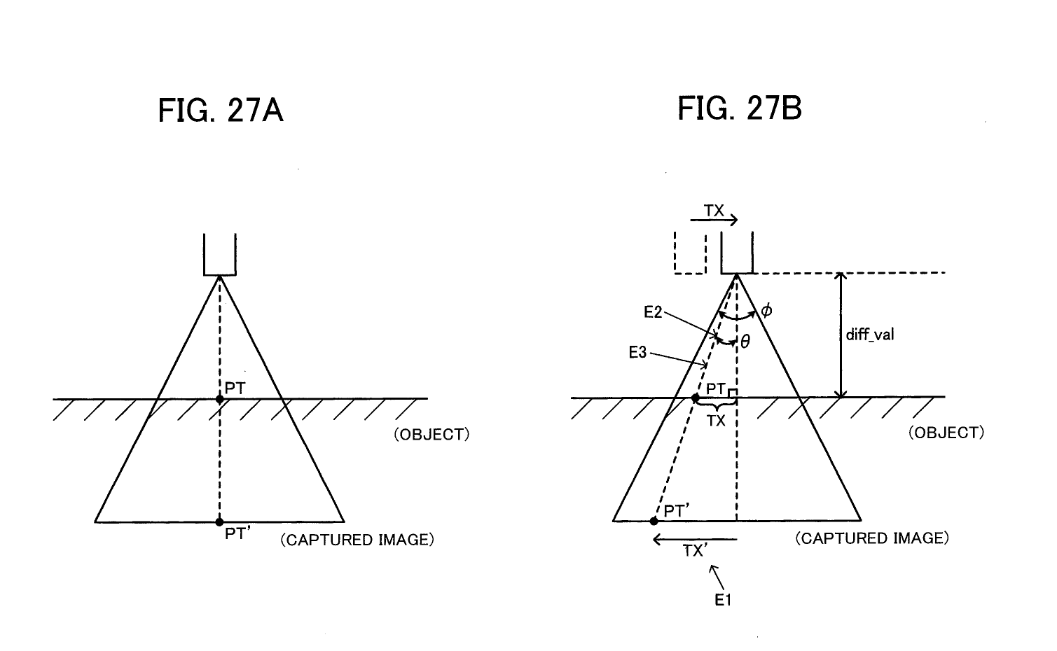

The average distance (diff_val) estimation process is described in detail below with reference to FIGS. 27A and 27B. The following description is given taking an example in which TY=0 for convenience. The angle of view is constant independently of the in-focus object plane position, and the magnification changes depending on the distance between the end of the imaging section 200 and the object.

As illustrated in FIG. 27A, a point PT of the object is displayed at a corresponding point PT′ of the image at the time t−1. As indicated by E1 in FIG. 27B, the corresponding point PT′ of the image has moved by a distance TX′ from the time t−1 to the time t. Since the angle of view φ of the imaging section 200 is known, the angle θ (see E2) can be determined from the distance TX′ on the image. Therefore, a right-angled triangle that has a distance TX on the object as a base is obtained (see E3), and the distance diff_val between the end of the imaging section 200 and the object can be determined.

Note that the average distance diff_val between the end of the imaging section 200 and the object cannot be estimated when the information (TX, TY) about the moving amount output from the control section 380 is (0, 0). In this case, a trigger signal that indicates that it is impossible to estimate the average distance is output to the magnification estimation section 342.

The process performed by the magnification estimation section 342 differs depending on the information output from the average distance estimation section 341. Specifically, when the average distance diff_val has been output from the average distance estimation section 341, the magnification estimation section 442 estimates the magnification Z″ using the following expression (21).

$${Z}^{″} = \frac{diff\_val}{diff\_org} \qquad ( 21 )$$

Note that the distance diff_org is the average distance between the end of the imaging section 200 and the object during normal observation. The distance diff_org may be the in-focus object plane position do during normal observation. or the user may set an arbitrary value as the distance diff_org via the external I/F section 500.

The magnification estimation section 342 outputs the magnification Z″ calculated using the expression (21) to the image extraction section 350c and the magnification storage section 343. The magnification Z″ in the preceding frame is stored in the magnification storage section 343.

When the trigger signal has been output from the average distance estimation section 341, the magnification estimation section 342 outputs the information stored in the magnification storage section 343 to the image extraction section 350c as the magnification Z″.

The above process makes it possible to implement stable shake canceling during magnifying observation using an optical system of which the in-focus object plane position is changed, even when the above in-focus object plane position detection means is not provided. Moreover, it is possible to suppress a situation in which the attention area is missed during magnifying observation.

According to the third configuration example, the image processing device may include a moving amount information acquisition section (i.e., the control section 380 that acquires moving amount information from the position sensor 280) that acquires moving amount information about the imaging section 200 (see FIG. 23). The imaging magnification calculation section (magnification calculation section 340c) may calculate the imaging magnification Z″ based on the moving amount information and the motion information. More specifically, the imaging magnification calculation section may calculate the distance diff_val between the imaging section 200 and the object based on the motion information (distance TX′) and the moving amount information (distance TX), and may calculate the imaging magnification Z″ based on the ratio of the distance diff_val to the reference distance diff_org (see FIG. 27B).

This makes it possible to calculate the imaging magnification Z″ of the imaging section 200 from the moving amount of the imaging section 200 that has been sensed by a motion sensor when using an endoscope apparatus that magnifies the object by moving the end of the imaging section 200 closer to the object.

### 10. Software

Although an example in which each section of the image processing section 300 is implemented by hardware has been described above, another configuration may also be employed. For example, a CPU may perform the process of each section. Specifically, the process of each section may be implemented by means of software by causing the CPU to execute a program. Alternatively, part of the process of each section may be implemented by software. Note that a software process may be performed on an image acquired in advance using an imaging device such as a capsule endoscope, instead of performing the shake canceling process on a moving image captured in real time.

When separately providing the imaging section, and implementing the process of each section of the image processing section 300 by means of software, a known computer system (e.g., work station or personal computer) may be used as the image processing device. A program (image processing program) that implements the process of each section of the image processing section 300 may be provided in advance, and executed by the CPU of the computer system.

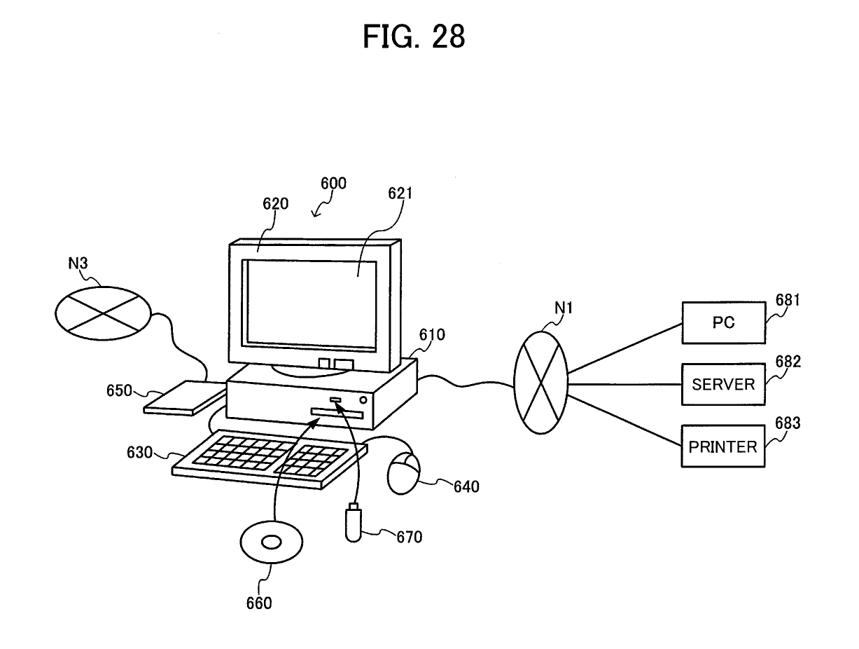

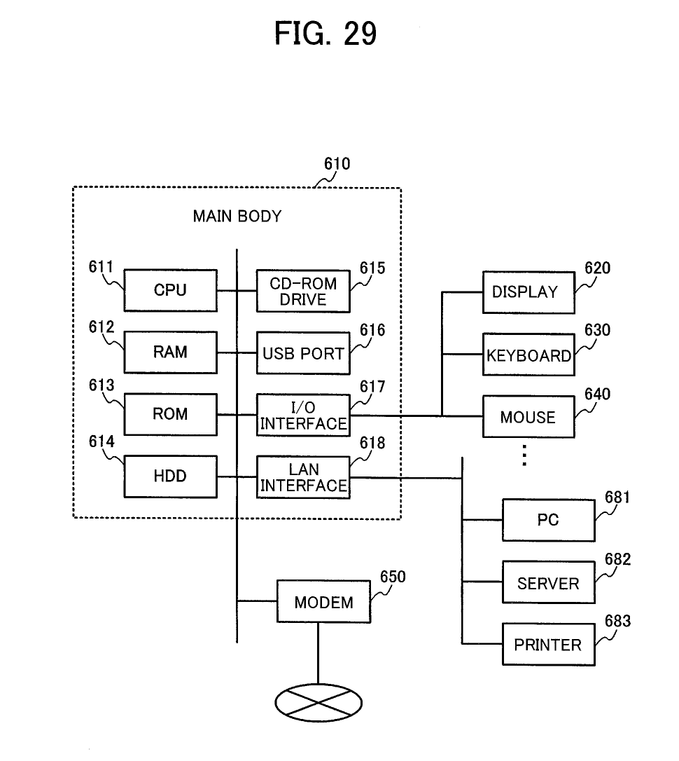

FIG. 28 is a system configuration diagram illustrating the configuration of a computer system 600 according to a modification. FIG. 29 is a block diagram illustrating the configuration of a main body 610 of the computer system 600. As illustrated in FIG. 28, the computer system 600 includes the main body 610, a display 620 that displays information (e.g., image) on a display screen 621 based on instructions from the main body 610, a keyboard 630 that allows the user to input information to the computer system 600, and a mouse 640 that allows the user to designate an arbitrary position on the display screen 621 of the display 620.

As illustrated in FIG. 29, the main body 610 of the computer system 600 includes a CPU 611, a RAM 612, a ROM 613, a hard disk drive (HDD) 614, a CD-ROM drive 615 that receives a CD-ROM 660, a USB port 616 to which a USB memory 670 is removably connected, an I/O interface 617 that connects the display 620, the keyboard 630, and the mouse 640, and a LAN interface 618 that is used to connect to a local area network or a wide area network (LAN/WAN) N1.

The computer system 600 is connected to a modem 650 that is used to connect to a public line N3 (e.g., Internet). The computer system 600 is also connected to a personal computer (PC) 681 (i.e., another computer system), a server 682, a printer 683, and the like via the LAN interface 618 and the local area network or the large area network N1.

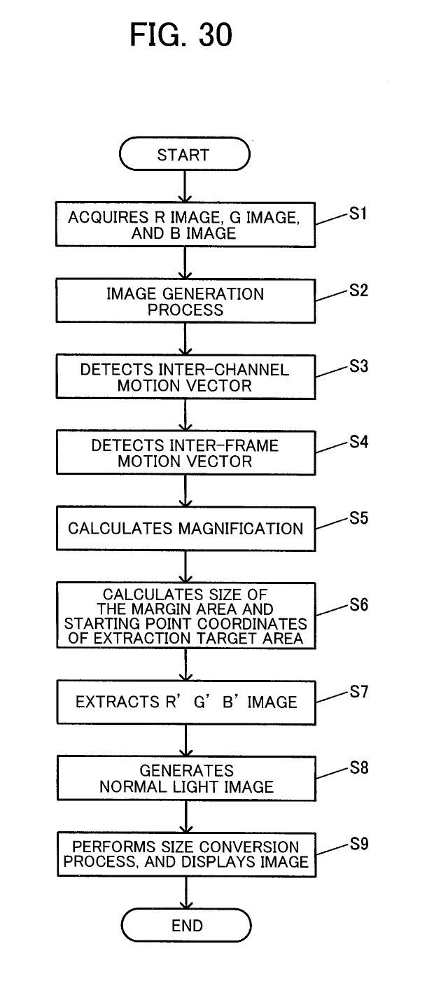

The computer system 600 implements the functions of the image processing device by reading an image processing program (e.g., an image processing program that implements a process described below with reference to FIG. 30) recorded in a given recording device, and executing the image processing program. The given recording device may be an arbitrary recording device that records the image processing program that can be read by the computer system 600, such as the CD-ROM 660, the USB memory 670, a portable physical device (e.g., MO disk, DVD disk, flexible disk (FD), magnetooptical disk, or IC card), a stationary physical device (e.g., HDD 614, RAM 612, or ROM 613) that is provided inside or outside the computer system 600, or a communication device that temporarily stores a program during transmission (e.g., the public line N3 connected via the modem 650, or the local area network or the wide area network N1 to which the computer system (PC) 681 or the server 682 is connected).

Specifically, the image processing program is recorded on a recording device (e.g., portable physical device, stationary physical device, or communication device) so that the image processing program can be read by a computer. The computer system 600 implements the functions of the image processing device by reading the image processing program from such a recording device, and executing the image processing program. Note that the image processing program need not necessarily be executed by the computer system 600. The invention may be similarly applied to the case where the computer system (PC) 681 or the server 682 executes the image processing program, or the computer system (PC) 681 and the server 682 execute the image processing program in cooperation.

A process performed when implementing the process of the image processing section 300 on an image acquired by the imaging section by means of software is described below using a flowchart illustrated in FIG. 30 as an example of implementing part of the process of each section by means of software.

As illustrated in FIG. 30, an R image, a G image, and a B image captured in time series are acquired (step S1). The image generation process is performed on the acquired images (step S2), and the inter-channel motion vector is detected from the images subjected to the image generation process (step S3). The inter-frame motion vector is then detected (step S4), and the magnification is calculated (step S5). The size of the margin area and the starting point coordinates of the extraction target area are calculated from the inter-channel motion vector, the inter-frame motion vector, and the magnification (step S6), and an R′G′B′ image is extracted (step S7). A normal light image is generated from the extracted R′G′B′ image (step S8). The size conversion process is performed on the normal light image, and the resulting image is displayed on the display section (step S9). The process is thus completed.

The above embodiments may also be applied to a computer program product that stores a program code that implements each section (e.g., inter-channel motion vector detection section, inter-frame motion vector detection section, magnification calculation section, and image extraction section) described above.

The term “computer program product” refers to an information storage device, a device, an instrument, a system, or the like that stores a program code, such as an information storage device (e.g., optical disk device (e.g., DVD), hard disk device, and memory device) that stores a program code, a computer that stores a program code, or an Internet system (e.g., a system including a server and a client terminal), for example. In this case, each element and each process according to the above embodiments are implemented by corresponding modules, and a program code that includes these modules is recorded on the computer program product.

The embodiments to which the invention is applied, and the modifications thereof have been described above. Note that the invention is not limited to the above embodiments and the modifications thereof. Various modifications and variations may be made without departing from the scope of the invention. A plurality of elements described in connection with the above embodiments and the modifications thereof may be appropriately combined to implement various configurations. For example, some of the elements described in connection with the above embodiments and the modifications thereof may be omitted. Some of the elements described in connection with different embodiments or modifications thereof may be appropriately combined. Specifically, various modifications and applications are possible without materially departing from the novel teachings and advantages of the invention.

Any term (e.g., magnification, translation amount, or specific area) cited with a different term having a broader meaning or the same meaning (e.g., imaging magnification, moving amount, or extraction target area) at least once in the specification and the drawings can be replaced by the different term in any place in the specification and the drawings.

What is claimed is:

1. An image processing device that processes an image acquired by an imaging section that enables magnifying observation, the image processing device comprising:
a motion information acquisition section that acquires motion information that indicates a relative motion of the imaging section with respect to an object;
an imaging magnification calculation section that calculates an imaging magnification of the imaging section; and
an image extraction section that extracts an image within a specific area from a captured image acquired by the imaging section as an extracted image,
the image extraction section setting a position of the specific area within the captured image based on the motion information, and setting a size of a margin area based on the imaging magnification and the motion information, the margin area being an area except the specific area in the captured image.

2. The image processing device as defined in claim 1,
the image extraction section setting the size of the margin area to a size that is proportional to the imaging magnification.

3. The image processing device as defined in claim 2,
the image extraction section setting the size of the margin area to a size obtained by multiplying a reference size by the imaging magnification, the reference size being a reference of the size of the margin area.

4. The image processing device as defined in claim 3,
the image extraction section updating the size of the margin area that has been set to the size obtained by multiplying the reference size by the imaging magnification based on the motion information.

5. The image processing device as defined in claim 4,
the image extraction section updating the size of the margin area based on an average value of the motion information within a given period.

6. The image processing device as defined in claim 5,
the image extraction section updating the size of the margin area with a size that is larger than the size set based on the imaging magnification when the average value of the motion information is larger than a first threshold value, and updating the size of the margin area with a size that is smaller than the size set based on the imaging magnification when the average value of the motion information is smaller than a second threshold value.

7. The image processing device as defined in claim 1,
the image extraction section updating the size of the margin area that has been set based on the imaging magnification based on the motion information.

8. The image processing device as defined in claim 7,
the motion information being a motion vector that indicates a motion of the object within the captured image, and
the image extraction section updating the size of the margin area based on an average value of an absolute value of the motion vector within a given period.

9. The image processing device as defined in claim 1,
the image extraction section resetting the specific area within the captured image when it has been determined that at least part of the specific area is positioned outside the captured image.

10. The image processing device as defined in claim 9,
the motion information being a motion vector that indicates a motion of the object within the captured image, and
the image extraction section resetting the position of the specific area within the margin area when a magnitude of the motion vector has exceeded the size of the margin area.

11. The image processing device as defined in claim 1, further comprising:
an in-focus object plane position information acquisition section that acquires in-focus object plane position information about the imaging section,
the imaging magnification calculation section calculating the imaging magnification based on the in-focus object plane position information when the imaging magnification is changed by changing a distance between the imaging section and the object.

12. The image processing device as defined in claim 11,
the imaging magnification calculation section calculating the imaging magnification based on a ratio of a reference in-focus object plane position to an in-focus object plane position indicated by the in-focus object plane position information.

13. The image processing device as defined in claim 1, further comprising:
an angle-of-view information acquisition section that acquires angle-of-view information about the imaging section,
the imaging magnification calculation section calculating the imaging magnification based on the angle-of-view information.

14. The image processing device as defined in claim 13,
the imaging magnification calculation section calculating the imaging magnification based on a ratio of tan(φ/2) to tan(φ/2) when a reference angle of view is φn, and an angle of view indicated by the angle-of-view information is φ.

15. The image processing device as defined in claim 1, further comprising:
a moving amount information acquisition section that acquires moving amount information about the imaging section,
the imaging magnification calculation section calculating the imaging magnification based on the motion information and the moving amount information.

16. The image processing device as defined in claim 15,
the imaging magnification calculation section calculating a distance between the imaging section and the object based on the motion information and the moving amount information, and calculating the imaging magnification based on a ratio of the calculated distance to a reference distance.

17. The image processing device as defined in claim 1,
the motion information acquisition section acquiring a motion vector that indicates a motion of the object within the captured image based on at least two captured images acquired at different times.

18. The image processing device as defined in claim 17,
the imaging section sequentially acquiring a first color signal image, a second color signal image, and a third color signal image in time series as the captured image,
the motion information acquisition section acquiring a motion vector that indicates a motion of the object between the first color signal image, the second color signal image, and the third color signal image as an inter-channel motion vector, and
the image extraction section extracting the extracted image from the first color signal image, the second color signal image, and the third color signal image based on the inter-channel motion vector.

19. The image processing device as defined in claim 1, further comprising:
a size conversion section that converts a size of the extracted image to a given size that can be displayed on a display section when the size of the extracted image changes corresponding to the imaging magnification.

20. The image processing device as defined in claim 19,
the imaging section acquiring a series of images that is a moving image as the captured image, and
the size conversion section converting a series of extracted images extracted from the series of images to have an identical size.

21. An endoscope apparatus comprising the image processing device as defined in claim 1.

22. The endoscope apparatus as defined in claim 21, further comprising:
a size conversion section that converts a size of the extracted image to a given size that can be displayed on a display section when the size of the extracted image changes corresponding to the imaging magnification; and
a display section that displays the extracted image of which the size has been converted to the given size.

23. A computer-readable storage device with an executable program stored thereon, wherein the program instructs a computer to perform steps of:
acquiring motion information that indicates a relative motion of an imaging section with respect to an object;
calculating an imaging magnification of the imaging section;
setting a position of a specific area within a captured image acquired by the imaging section based on the motion information, and setting a size of a margin area based on the imaging magnification and the motion information, the margin area being an area except the specific area in the captured image; and
extracting an image within the specific area from the captured image as an extracted image.

24. An image processing method comprising:
acquiring motion information that indicates a relative motion of an imaging section with respect to an object;
calculating an imaging magnification of the imaging section;
extracting an image within a specific area from a captured image acquired by the imaging section as an extracted image; and
setting a position of the specific area within the captured image based on the motion information, and setting a size of a margin area based on the imaging magnification and the motion information, the margin area being an area except the specific area in the captured image.
